(19) United States
(12) Patent Application Publication
(10) Pub. No.: US 2004/0142449 A1
(43) Pub. Date: Jul. 22, 2004

(54) Replication-competent molecular clones of porcine endogenous retrovirus class a and class b derived from pig and human cells

(76) Inventors: Ralf R. Tonjes, Weiterstadt (DE); Ulrich Krach, Darmstadt (DE); Marcus Niebert, Mulheim/Ruhr (DE)

Correspondence Address: FISH & RICHARDSON PC, 225 FRANKLIN ST, BOSTON, MA, 02110, US

(21) Appl. No.: 10/471,220

(22) PCT Filed: Mar. 11, 2002
(86) PCT No.: PCT/EP02/02656

(30) Foreign Application Priority Data
Mar. 9, 2001 (DE) 101 11 433.8

(51) Int. Cl. C12P 19/34; C12N 9/22; C12N 7/00; C07K 14/155

(52) U.S. Cl. 435/235.1; 435/199; 530/350

(57) ABSTRACT

The present invention relates to functional, replication-competent full-length proviral PERV-A and PERV-B clones isolated directly from the pig genome, i.e. “native” PERV and allows the comparison of proviral PERV sequences from different origins on the molecular, structural and cellular level.

The present invention relates to replication-competent molecular clones of porcine endogenous retrovirus (PERV).

### BACKGROUND OF THE INVENTION

The better understanding of the cellular and molecular basis of transplant rejection and the generation of transgenic donor animals bearing genes that mediate protection towards rejection (Bach, F. H. et al., 1996, Proc. Natl. Acad. Sci. USA 93:7190-7195) have stimulated approaches to use xenotransplantation, i.e. the therapeutic use of animal cells, tissues and organs, to overcome the shortage of allogeneic transplants (Dorling, A. et al., 1997, Lancet 349:867-871). Pigs are preferred as donors forxenotransplants due to related physiology, ease of breeding and for ethical reasons (Fishman, J. A. 1994, Xenotransplantation 1:47-57). Up to now, clinical trials included the implantation of fetal neuronal tissue as a therapy for Parkinson's and Huntington's disease (Deacon, T., J. et al., 1997, Nat. Med. 3:350-353; Fink, J. S. et al., 2000, Cell Transplantation 9:273-278), the implantation or infusion of pancreatic islet cells as a treatment for insulin-dependent diabetes mellitus (Groth, C. G. et al, 1994, Lancet 344:1402-1404), extra corporeal kidney perfusion (Breimer, M. E. et al., 1996, Xenotransplantation 3:328-339), bioartificial liver devices (Mullon, C. and Z. Pitkin, 1999, Exp. Opin. Invest. Drugs 8:229-235) and perfusion through or the implantation of whole liver preparations as a treatment for hepatic failure (Chari, R. S. et al., 1994, N Engl. J. Med. 331:234-237; Cramer, D. V., 1995, Transplant. Proc. 27:80-83).

Major concerns have been raised in regard of the possibility to introduce new microbial agents from the animal into the recipient leading to xenozoonosis (Allan, J. S. 1996, Nat. Med. 2:18-21; Fishman, J. A. 1997, Kidney Int. 51(Suppl. 58):41-45; Michaels, M. G. and R. L. Simmons, 1994, Transplantation 57:1-7; Stoye, J. P. and J. M. Coffin 1995, Nat. Med. 1:1100). In this respect, breeding and keeping pigs under specific-pathogen-free (SPF) condition is considered to reduce the risk of transmitting exogenous agents. However, these methods are not appropriate to avoid the presence of viruses that are germline-transmitted, i.e. porcine endogenous retroviruses (PERV) (Patience, C. et al., 1997, Nat. Med. 3:282-286), and DNA viruses that can persist without symptoms in their natural host and are transmitted via intrauterine or transplacentar pathways, e.g. herpesviruses (Ehlers, B. et al., 1999, J Gen. Virol. 80:971-978).

Referring to PERVs, approximately 50 integration sites exist in the genome of different pig breeds (Akiyoshi, D. E. et al., 1997, Nature 389:681-682; Patience, C. et al., 1997, Nat. Med. 3:282-286) and at least three classes of PERV are known (LeTissier, P. et al., 1997, Nature 389:681-682, Takeuchi, Y. et al., 1998, J. Virol. 72:9986-9991). Those classes, named PERV-A, -B, and -C, display high sequence homology in the genes for the group specific antigens (gag) and the polymerase pol) but differ in the envelope (env) genes which determine the host range. Recently, a new class of PERV env gene, designated Env-D, has been described (WO 00/11187).

Recent reports demonstrated that PERV which are released from different pig cell lines are able to infect human cells in vitro (Martin, U. et al., 1998, Lancer 352:692-694; Wilson, C. A. et al., 2000, J. Virol. 74:49-56; Wilson, C. A. et al., 1998, J. Virol. 72:3082-3087). PERV-C, also designated PERV-MSL (Akiyoshi, D. E. et al., 1998, J. Virol. 72:4503-4507), is ecotropic compared to PERV-A and PERV-B which are polytropic as deduced from pseudotype experiments utilizing the corresponding env genes for MLV vectors (Takeuchi, Y. et al., 1998, J. Virol. 72:9986-9991). In addition, the cloning of full-length, replication-competent PERV-B proviral sequences derived from infected human 293 cells (293 PERV-PK; Czaudema, F. et al., 2000, J. Virol. 74:4028-4038) has been recently reported. These data, in addition to the characterization of a PERV-C proviral sequence (PERV-MSL; Aidyoshi, D. E. et al., 1998, J. Virol. 72:4503-4507), demonstrates that the pig genome harbors intact proviruses similar to those found in several other species including humans (Tönjes, R. R. et al., 1999, J. Virol 73:9187-9195).

Retrospective investigation of 160 patients that have been treated with porcine cells and tissues showed no evidence for transmission of PERV (Paradis, K. G. et al., Science 285:1236-1241) but no long-term transplantation of a whole vascularized organ has been attempted so far. However, recent studies utilizing immunodeficient NOD/SCID mice revealed PERV infection in several tissue compartments after transplantation of pig pancreatic islets (Van der Laan, L. J. W. et al., 2000, Nature, 407:90-94).

From the above, it is evident that such vertically transmitted endogenous retroviruses pose an infectious risk in the course of pig-to-human transplantation of cells, tissues and organs. Expression and possible replication of PERV, even at low levels, has a major implication on the use of pig organs and tissues in the course of xenotransplantation. Therefore, it is highly desirable to generate PERV-free strains of pigs for xenotransplantation.

One prerequisite for the generation of PERV-free pig strains is the identification of “native” replication-competent retroviruses in the pig genome. Identification of said “native” retroviruses would enable the screening of different pig breeds for the presence of infectious PERV and accordingly, the identification of pig breeds which produce lower levels of PERV or which are devoid of individual proviruses due to polymorphisms.

So far, “native” replication-competent PERV have not yet been isolated from the pig genome. Thus, it is not possible to genetically screen pig breeds for the presence of infectious PERV, which greatly increases the infectious risk for a patient receiving a xenotransplant of porcine origin.

In order to solve this problem, it is highly desirable to isolate “native” replication-competent PERV from the porcine genome, to fully characterize and chromosomally assign those proviruses and/or to provide integration site-specific sequence information for screening purposes.

The present invention provides for the first time functional, replication-competent full-length proviral PERV-A and PERV-B clones isolated directly from the pig genome, i.e. “native” PERV and allows the comparison of proviral PERV sequences from different origins on the molecular, structural and cellular level. In particular, the present invention describes the cloning and characterization of PERV-A and PERV-B proviral sequences derived from the porcine kidney cell line PK15 (Patience, C. et al., 1997, Nat Med. 3:282-286).

Furthermore, this invention describes the isolation of “native” infectious PERV-A and PERV-B clones derived from a porcine bacterial artificial library (Rogel-Gaillard et al., 1999, Cytogenet. Cell Genet. 85:205-211), which further enabled the mapping of PERV proviral sequences to chromosome locations of one specific pig breed.

### DETAILED DESCRIPTION OF THE INVENTION

This invention describes for the first time “native” replication-competent molecular clones of porcine endogenous retroviruses (PERV). The invention is further directed to a method which enables the identification and subsequent isolation of such clones. Furthermore, the present invention comprises nucleic acid sequences encoding replication-competent PERV and methods for detecting the presence of replication-competent PERV in a biological sample. Furthermore, the invention provides pig genomic sequences which flank genomic integration sites of the replication-competent PERV of this invention.

In one embodiment, the invention relates to a replication-competent molecular clone of porcine endogenous retrovirus (PERV), wherein said molecular clone was isolated from porcine cells and is replication-competent upon transfection into susceptible cells.

As used herein, the term “replication-competent” denotes the ability of a clone to yield, upon transfection/infection of susceptible cells, productive infection of said cells, i.e. the infected cells release viral particles. Examples of cells susceptible for PERV infection include human 293 cells (Patience, C. et al., 1997, Nat. Med. 3:282-286, Takeuchi, Y., C. et al., 1998, J. Virol. 72:9986-9991) and HeLa cells (ECACC 93021013), as well as canine D17 cells and feline PG4 cells that can be obtained from the European Collection Of Cell Cultures ECACC).

In a preferred embodiment of the invention, the replication-competent molecular clone is a PERV-A or PERV-B clone. Isolation of such clones can be accomplished using the method according to the present invention. Thus, in another embodiment, the invention relates to a method for isolating a replication-competent molecular clone of PERV, comprising the steps of a) establishing a DNA library from a porcine cell line, wherein said cell line releases infectious PERV particles, b) screening said DNA library with a PERV-specific pro/pol probe, c) isolating clones containing proviral sequences which react with the PERV-specific pro/pol probe from said DNA library, d) analyzing said proviral sequences from said DNA librauy with PCR employing PCR primers specific for PERV-A and PERV-B env genes, and e) determining the presence of a proviral ORF in the isolated proviral sequences by protein truncation test (PTT; Roest, P. A. M. et al., Hum. Molec. Genet. 2:1719-1721; Tönjes, R. R. et al., 1999, J. Virol. 73:9187-9195) using the TNT T7 Quick coupled Transcription/Translation System (Promega, Mannheim, Germany) according to the manufacturer's instructions.

In a preferred embodiment, after step (e) of the above-referenced method, the replication-competence of the isolated clone is determined by f) transfecting susceptible cells with the isolated clone, and g) detecting productive infection of susceptible cells by indirect immunofluorescence analysis using a PERV-specific Gag p10 antiserum (Krach, U. et al., 2000, Xenotransplantation 7:221-229) and determining reverse transcriptase activity in the supernatants of the infected susceptible cells (RT assay; Czauderna F. et al., 2000, J. Virol. 74:4028-4038) employing the C-type RT activity assay (Cavidi Tech Ab, Uppsala, Sweden) according to the manufacturer's instructions (protocol B).

In a further aspect, the invention relates to the generation of PERV-specific antisera. In particular, the invention relates to a PERV-specific p30 or p15E antiserum. Said antisera can be used for detecting productive infection of susceptible cells (see FIGS. 11, 12 and 13)

In a preferred embodiment of the invention, the porcine cell line employed for establishing a DNA library is PK15 (Patience, C. et al., 1997, Nat. Med. 3:282-286).

In a particularly preferred embodiment of the invention, the replication-competent molecular clone of PERV-A or PERV-B is PK15-PERV-A(58) or PK15-PERV-B(213), respectively. PK15-PERV-A(58) is encoded by a nucleic acid sequence corresponding to SEQ ID NO:1 (see also FIG. 14). PK15-PERV-B(213) is encoded by a nucleic acid sequence corresponding to SEQ ID NO:2 (see also FIG. 15).

Said clones were isolated according to the method of the present invention as follows: First, a DNA library was established from the porcine cell line PK15 which releases infectious PERV particles (Patience, C. et al., 1997, Nat. Med. 3:282-286). The library was screened with a PERV-specific pro/pol probe to isolate “native” proviral sequences. After three rounds of screening, 68 clones were purified to homogeneity. Differentiation of these clones by PCR utilizing primers specific for env-A and env-B genes revealed 41 PERV-A clones and 10 PERV-B clones, respectively. The remaining 17 clones yielded neither env-A nor env-B amplificates. Furthermore, these clones did not comprise env class C or D sequences and were thus considered as deficient of the appropriate env gene sequences and were excluded from further analysis.

According to the method of the present invention, the presence of a proviral ORF in the isolated clones was subsequently investigated by PTT analyses (Roest, P. A. M. et al., 1993, Hum. Molec. Genet. 2: 1719-1721; Tönjes, R. R. et al., 1999, J. Virol. 73:9187-9195). While most of the isolated clones were truncated in either one or more of the three ORFs, three class A clones, λPK15-PERV-A(42), λPK15-PERV-A(45) and λPK15-PERV-A(58), and one class B clone, λPK15-PERV-B(213), demonstrated all three reading frames. Restriction enzyme analyses and partial sequencing suggested that the three PERV-A sequences are identical. Thus, only clone λPK15-PERV-A(58) was chosen for further experiments and was designated pPK15-PERV-A(58) after subcloning of the NotI insert from bacteriophage λ into pBS. Clone λPK15-PERV-B(213) was further analyzed after subcloning of the corresponding λ insert, yielding plasmid pPK15-PERV-B(213).

Summarizing, the method according to the present invention yielded clones PK15-PERV-A(58) and PK15-PERV-B(213). In accordance with the method of the present invention, the capacity of the viruses PK15-PERV-A(58), and PK15-PERV-B(213) to infect susceptible cell lines was revealed by detection of Gag expression and viral particles in cell-free supernatants of infected cells using RT assays (see FIGS. 4, 5, 6).

The clone 293-PERV-A(42) which had been isolated from a human 293 cell line productively infected with PERV (293-PERV-PK) (Czaudema F. et al., 2000, J. Virol. 74:4028-4038), was analyzed accordingly. Since clone 293-PERV-A(42) was cloned from infected human cells, it is not a “native”, but a “humanized” PERV clone.

The PERV clones described here showed levels of RT activity on 293 cells of 15 mU/ml for 293-PERV-A(42), and 4 mU/ml for PK15-PERV-B(213); FIG. 7). The most susceptible cell line for 293-PERV-A(42) was the feline cell line PG4 that demonstrated RT activities of up to 500 mU/ml (FIG. 7A). Furthermore, lower level but transient activity was found for canine D17 cells (FIG. 7A), which is in accordance with a previously published host range study (Takeuchi, Y. et al., 1998, J. Virol. 72:9986-9991).

PK15-PERV-A(58) showed a significantly lower activity of up to three logarithmic scales compared to 293-PERV-A(42) and, except for 293 cells, only transient activity barely above background was observed for the other cell lines investigated (FIG. 7B).

Infection studies with clone PK15-PERV-B(213) revealed only low activities on HeLa cells (24 mU/ml until day 50) and transient activities on 293 cells (4 mU/ml on day 21). These findings are different from previous results where efficient entry of pseudotyped MLV was mediated by PERV-B env (Takeuchi, Y. et al., 1998, J. Virol. 72:9986-9991). All other cell lines tested revealed only background activities.

Analysis of Gag expression in infected cell lines by immunofluorescence using PERV-specific antisera revealed patterns similar to those described previously for PERV-infected cell lines (Krach, U. et al., 2000, Xenotransplantation 7:221-229) (FIG. 5).

In a further embodiment, the invention relates to replication-competent molecular clones of PERV obtained by another method than the one previously described. Thus, in said embodiment, the invention relates to replication-competent PERV-A and PERV-B clones derived from a porcine bacterial artificial chromosome library constructed from primary fibroblasts derived from large white pigs (Rogel-Gaillard et al., 1999, Cytogenet. Cell Genet. 85:205-211). In a preferred embodiment, the invention relates to the PERV-A clone PERV-A(Bac-130A12) and to the PERV-B clone PERV-B(Bac-192B9).

PERV-A(Bac-130-A12) is encoded by a nucleic acid sequence corresponding to SEQ ID NO:3 (FIG. 16). PERV-B(3α-192B9) is encoded by a nucleic acid sequence corresponding to SEQ ID NO:4 (FIG. 17).

The replication-competence of said clones could be demonstrated by indirect immunofluorescence analysis of transfected or infected cell lines using a PERV-specific antiserum against Gag p 10. As shown for 293 cells (FIG. 6), Gag expression in an increasing number of cells was observed for clone PERV-A(Bac-130A12) after incubation with p10 antiserum 7 days, 10 days and 35 days p.t. which indicated the replication-competence of this provirus. For PERV-B(Bac-192B9), immunoreactivity was detected for up to 10 days p.t., but diminished when the cells were cultured for longer periods of time (FIG. 6). The initial immunoreactivity of cells transfected with PERV-B(Bac-192B9) can be explained by transient LTR-mediated expression of Gag shortly after transfection due to the deficiency of this clone to establish productive infection (see below).

The results obtained by immunofluorescence analyses were confirmed by measuring the activity of the viral RT. Cell-free culture supernatants from human HeLa and 293 cells transfected and/or infected with the clones PERV-A(Bac-130A12) and PERV-B(3α-192B9), respectively, were collected up to 45 days post transfection (p.t)/post infection (p.i.) (FIG. 8). For clone PERV-A(Bac-130A12), RT activity was found on Hela cells (up to 190 mU/ml[UK1]) (FIG. 8). No RT activity was observed for clone PERV-B(Bac-192B9).

In addition to the provision of “native” replication-competent molecular clones of PERV, the invention further relates to nucleic acid sequences encoding replication-competent molecular clones of PERV-A and PERV-B. Particularly preferred are the nucleic acid sequences identified by SEQ ID NO:1, SEQ ID NO:2, SEQ ID NO:3 and SEQ ID NO:4, which encode the molecular clones PK15-PERV-A(58), PK-15-PERV-B(213), PERV-A(13α-130A12) and PERV-B(Bac-192B9), respectively.

The proviral sequences PK15-PERV-A(58) (SEQ ID NO:1), PK15-PERV-B(213) (SEQ ID NO:2), PERV-A(Bac-130A12) (SEQ ID NO:3) and PERV-B(1α-192B9) (SEQ ID NO:4) are 8918, 8763, 8918 bp and 8840 bp in length, respectively. The LTRs are 668 bp (293PERV-A(42)), 707 bp (PK15-PERV-A(58)) and 630 bp (PK15-PERV-B(213)) long and characterized by the presence of different numbers as well as a different structural assembly of 18 bp and 21 bp subrepeats (FIG. 3). PERV-A(Bac-130A12) and PERV-B(Bac-192B9) bear LTRs of 702 bp and 668 bp, respectively. Although PERV-B(Bac-192B9) is a class B PERV sequence it bears an LTR structure similar to one only found in a type A PERV until now.

Comparison of nucleotide and amino acid sequences revealed that PK15-PERV-B(213) is highly homologous but distinct from a previously described clone PERV-B(43) (Czauderna F. et al., 2000, J. Virol. 74:4028-4038). PK15-PERV-A(58) demonstrates close homology to PERV MSL in LTR, gag and pro/pol (gag, 97.6%; pro/pol, 97.5%) with exception of env (69.3%) for which PK15-PERV-A(58) demonstrates closer relationship to 293-PERV-A(42) (FIG. 2C) sequences. [UK2] The overall lower level of homology of PK15-PERV-A(58) compared to 293-PERV-A(42) (Table 1) is reflected by the phylogenetic distances of Gag and Pro/Pol (FIGS. 2A, B). From these data, it appears that PK15-PERV-A(58) forms a major group with PERV-MSL, irrespective of the env sequence.

The LTR of clone PERV-A(Bac-130A12) is 702 bp long, while the gag gene starts at nucleotide (nt) 1153 and is colinear with the pro/pol open reading frame (ORF) (nt 2728-6309). The stop codon at nt 2727 separating both genes is suppressed by a tRNA_{gin} as described previously (Akiyoshi et al., 1998, J. Virol. 72:45034507; Czauderna et al., 2000, J. Virol. 74:4028-4038). The env gene partially overlaps with pro/pol and forms a new ORF (nt 6185-8149). Clone PERV-A(Bac-130A12) has been chromosomally assigned and maps to 1q2.4.

PERV-B(Bac-192B9) shows a similar structure bearing an LTR of 668 bp and gag (nt 1115-2689), pro/pol (nt 2690-6277) and env (nt 8173-8123) genes, respectively. However, two stop codons at nt 4687 and nt 5251 within the pro/pol sequence disrupt the ORF and, as a consequence, prevent this clone from replication (FIG. 6). The chromosomal location of PERV-B(Bac-192B9) is 7p1.1. Hence, this provirus maps to the swine leukocyte antigen (SLA) Rogel-Gaillard et al., 1999, Cytogenet. Cell Genet. 85:205-211).

Sequences of PERV-A(Bac-130A12) and PERV-B(13α-192B9) showed close relationship to proviral PERV sequences described previously. PERV-A(Bac-130A12) is almost identical to PK15-PERV-A(58) (Krach et al., 2000, Xenotransplantation 7:221-229) demonstrating homologies of approximately 99% for the LTRs and the viral genes. However, both clones appear to map to different chromosomal locations as deduced from the flanking sequences. PERV-A(Bac-130A12), in comparison to 293-PERV-A(42) (Czauderna et al., 2000, J. Virol. 74:4028-4038; Krach et al., 2000, Xenotransplantation 7:221-229), shows slightly lower homologies of approximately 95% within the retroviral genes and a completely different LTR structure. PERV-B(Bac-192B9) demonstrates high homology (approximately 98%) to clone 293-PERV-B(33) (Czaudema et al., 2000, J. Virol. 74:4028-4038), however, the LTR of this provirus is similar to that of class A clone 293-PERV-A(42) which bears a characteristic 39 bp repeat structure in U3 (Czauderna et al., 2000, J. Virol. 74:4028-4038).

The polymorphisms found in 293-PERV-A(42), PK 15-PERV-A(58) and PK15-PERV-B(213) neither have an impact on the highly conserved motifs in pro/pol for mammalian type C retroviruses (Table 2) nor, in the case of PK15-PERV-A(58), on the regions in the env genes which are important for the determination of the host range (VRA, VRB, and PRO) (LeTissier, P. et al., 1997, Nature 389:681-682).

The invention is further directed to sequences derived from the pig genome flanking the proviral integration sites.

The sequences of clones PERV-A(Bac-130A12), PERV-A(151B10), PERV-A(Bac-463H12) and PERV-B(Bac-192B9) were determined displaying proviruses of 8918 bp, 8882 bp, 8754 bp and 8840 bp, respectively. While the sequence of the LTRs and viral genes were determined seperately, they were assembled for this analysis.

The gag gene of clone PERV-A(Bac-130A123) ranges from nt 1153 to nt 2727 and the pro/pol ORF is located in the same reading frame (nt 2875-6309). The env gene forms the third ORF (nt 6185-8149). Clone PERV-A(Bac-130A12) has been chromosomally assigned and maps to 1q2.4 (Rogel-Gaillard et al., 1999). The structure of the other clones is similar as given in the following paragraph:

Clone PERV-A(3α-151B10). gag: nt 1148-2711, pro/pol: nt 2859-6272, env: nt 6148-8112, position: 1q2.3

Clone PERV-A(Bac-463H12). gag: nt 1077-2660, pro/pol: nt 28326242, env: nt 6118-8100, position: 3p1.5

Clone PERV-B(Bac-192B9). gag: nt 1115-2689, pro/pol: nt 2837-6277, env: nt 8173-8123, position: 7p1.1.

Two stop codons at nt 4687 and nt 5251 within the pro/pol sequence disrupt the open reading frame and, as a consequence, prevent this clone from replication.

The proviral sequences of clones PERV-A(Bac-130A12), PERV-A(151B10), PERV-A(Bac-463H12) and PERV-B(Bac-192B9) have been deposited in Genbank (accession numbers AJ279056, AF435967, AF435966 and AJ279057, respectively).

Genomic flanking sequences were determined by inverse PCR. These sequences allow the identification of the respective proviruses within the porcine genome by simple PCR techniques, as the flanking sequences are unique for every provirus.

Further to the elucidation of the nucleic acid sequence of PK15-PERV-A(58) (SEQ ID NO:1), PK15-PERV-B(213) (SEQ ID NO:2), PERV-A(Bac-130A12) (SEQ ID NO:3) and PERV-B(Bac-192B9) (SEQ ID NO:4), the flanking sequences of said clones in the pig genome were determined.

The nucleic acid sequences corresponding to the 5′-and 3′-flanking sequences of PK15PERV-A(58) are identified by SEQ ID NOs:5 and 6, respectively. The nucleic acid sequences corresponding to the 5′- and 3′-flanking sequences of PK15-PERV-B(213) are identified by SEQ ID NOs:7 and 8, respectively. The flanking sequences of PK15-PERV-A(58) and PK15PERV-B(213) are also shown in FIGS. 18A, 18B and 19A, 19B, respectively.

Clone PERV-A(Bac-130A12) has been chromosomally assigned and maps to chromosome 1q2.4 (Rogel-Gaillard et al., 1999, Cytogenet. Cell Genet. 85:205-211). The nucleic acid sequences corresponding to the 5′- and 3′-flanking sequences of PERV-A(Bac-130A12) are identified by SEQ ID NOs: 9 and 10 (FIGS. 20A and 20B), respectively.

The chromosomal location of PERV-B(Bac-192B9) is 7p 1.1, and therefore maps to the SLA.

The nucleic acid sequences corresponding to the 5′-and 3′-flanking sequences of PERV-B(Bac-192B9) are identified by SEQ ID NOs: 11 and 12 (FIGS. 21A and 21B), respectively.

The nucleic acid sequence corresponding to the 3′-flanking sequence of PERV-A (Bac463H12) is identified by SEQ ID NO: 13 (FIG. 22) and the nucleic acid sequence corresponding to the 3′-flanking sequence of PERV-A (Bac-15 B10) is identified in SEQ ID NO:14 (FIG. 23).

The data of the present invention suggest that the pig genome harbors a limited number of infectious PERV sequences at particular integration sites. Thus, the flanking sequences according to the present invention can be used for the detection of specific and functional PERV.

In a preferred embodiment of the present invention, oligonucleotides comprising 12-60 nucleotides, preferably 1540 nucleotides and most preferably 15, 16, 17, 18, 19 or 20 to 30 nucleotides of the 5′-and/or 3′-flanking sequences of PK15-PERV-B(213), of PK15-PERV-A(58), of PERV-A(13α-130A12) and/or of PERV-B(Bac-192B9) or oligonucleotides which are complementary to the above-mentioned flanking sequences and comprise 12-60 nucleotides, preferably 1540 nucleotides and most preferably 15, 16, 17, 18, 19 or 20 to 30 nucleotides or which hybridize to the above-mentioned flanking sequences and comprise 17-60 nucleotides, preferably 17-40 nucleotides and most preferably 18, 19 or 20 to 30 nucleotides are used in a method for detecting integrated PERV. Examples of methods of detection of integrated PERVs according to the present invention are PCR and Southern blot analysis.

The term “hybridize” referred to herein means that the oligonucleotides of the invention selectively hybridize to nucleic acids strands under hybridization and wash conditions that minimize appreciable amounts of detectable binding to nonspecific nucleic acids. High stringent conditions can be used to achieve selective hybridization conditions as known in the art and discussed herein. When using oligonucleotides which hybridize to the above mentioned flanking sequences of the present invention the oligonucleotides are at least 17 nucleotides, preferably 18, 19 or 20 to 30 nucleotides long and have a homology of at least about 70%, about 90%, about 95%, about 98% or 100% to the complementary sequences of the above mentioned flanking sequences.

In another aspect, this invention provides a method for detecting the presence of replication-competent PERV in a sample, comprising detecting a nucleic acid sequence corresponding to SEQ ID NO:1, SEQ ID NO:2, SEQ ID NO:3 or SEQ ID NO:4 or parts thereof.

A further aspect of the present invention relates to a polypeptide derived from the Gag and/or the Env sequence encoded by the nucleic acid sequence of SEQ ID NOs: 1, 2, 3 or 4.

In another embodiment, the present invention relates to vaccines for immunizing a host against a replication-competent PERV, comprising an effective amount of a polypeptide derived from the Gag and Env sequences encoded by the nucleic acid sequence of SEQ ID NOs: 1, 2, 3 or 4.

In yet another aspect, the present invention relates to the production of PERV-free pigs. Based on the identification of “native” replication-competent retroviruses in the pig genome according to the present invention, it is now possible to screen different pig breeds for the presence of specific infectious PERVs and accordingly to identify pig breeds which are PERV-free.

The invention is further illustrated by the following figures.

### FIGURES

FIG. 1. Structures of 293-PERV-A(42), PK15-PERV-A(58), and PK15-PERV-B(213). Proviral sequences of 293-PERV-A(42), PK15-PERV-A(58) and PK15-PERV-B(213) are 8849 bp, 8918 bp and 8763 bp in length, respectively.

Genes are shown as open boxes and first and last nucleotide of LTR and genes are given (numbers in parentheses, PK15-PERV-A(58) and PK15-PERV-B(213)). Arrows indicate the transcriptional start site (cap), the primer binding site (PBS), splice donor (SD) and splice acceptor (SA), and the poly(A) addition site (@(A). The nt positions correspond to molecular clone 293-PERV-B(33) (Czaudema F. et al., 2000, J. Virol. 74:4028-4038) (Accession No. AJ133816).

FIG. 2. Phylogenetic relationship of PERV proteins.

Phylograms are based on fill-length open reading frames for Gag (A), Pro/Pol (B), and Env (C) (see also Table 1). Relative distances are indicated by scale bars (0,1 indicates 10% divergence). Phylograms were generated using Phylip 3.574c and the Prodist and Neighbor programs (http://evolution.genetics.washington.edu/phylip.htnl).

FIG. 3. Schematic structure of the 5′-LTR of 293-PERV-A(42), PK15-PERV-A(58), and PK15-PERV-B(213). LTRs are 668 bp (293-PERV-A(42)), 702 bp (PK15-PERV-A(58)), and 630 bp (PK15-PERV-B(213)) long. Repeat boxes consisting of 18-bp and 21-bp subrepeats are indicated as black and gray boxes.

FIG. 4. Proviral integration of PERV.

Detection of a 729 bp pro/pol amplification product by PCR. Lane 1, 7, and 11, 293 cells; lane 2, 8 and 12, HeLa cells; lane 3, 9 and 13, D17 cells; lane 4, 10 and 14, PG-4 cells; lane 5, molecular weight marker, lane 6 positive control; lane 15, water control. Lane 1 to lane 4, PK15-PERV-B(213); lane 7 to lane o, 293-PERV-A(42); lane 11 to lane 14, PK15-PERV-A(58).

FIG. 5. Indirect immunofluorescence analysis of HeLa cells infected with 293-PERV-A(42)(panel A) and 293 cells infected with PK15-PERV-A(58) (panel B). Cells were incubated with PERV Gag p10 antiserum (Krach, U. et al.,2000, Xenotransplantation 7:221-229). Magnification 400×.

FIG. 6. Expression of clones PERV-A(Bac-130A12) and PERV-B(Bac-192B9). Detection of PERV Gag by indirect immunofluorescence assay at different time points after transfection of BAC DNA using an antibody against p10. A-C, 293 cells transfected with PERV-A(Bac130A12), 7, 21 and 35 days post transfection (p.t.), respectively. D-F, 293 cells transfected with PERV-B(Bac-192B9), 7, 21 and 35 days p.t., respectively.

FIG. 7. Replication of 293-PERV-A(42) and PK15-PERV-A(58).

RT activity in cell-free culture supernatants of 293-PERV-A(42) (panel A) and PK15-PERV-A(58) (panel B) infected cells. Cell lines 293, HeLa, D17 and PGA were studied for up to 51 days post infection (post infection, p.i.). MoMLV RT was used as a standard.

FIG. 8. Detection of reverse transcriptase activity in cell-free culture supernatants of HeLa cells upon transfection of Bac DNA of clones PERV-A(Bac-130A12) and PERV-B(13ac192B9).

FIG. 9. Localization and amino acid sequences of PERV peptides used for immunizations of rabbits.

Positions of peptides in protein and gene sequences, respectively, are denoted. Positions refer to clone PERV-B(33)/ATG (Czaudema et al., 2000). Aa, amino acid, nt, nucleotide.

FIG. 10. Immunoblotting using α-p30U and α-p15E.

Lanes 1 and 3, lysate of cell line 293 PERV-PK; lanes 2 and 4, sucrose gradient purified virus particles. Lanes 1 and 2, incubation with α-p30U antiserum; Lanes 3 and 4, α-p15E antiserum. Arrows denote p30 protein (lane 2) and the Env precursor protein (p73, lane 3) and the glycosylated Env (p90^{gly}, lane 3) with apparent molecular masses of 73 kDa and 90 kDa, respectively.

FIG. 11. Indirect immunofluorescence analysis of PERV Gag expression using α-p30U antiserum.

Panels A, C and E, α-p30U antiserum; panels B, D and F, preimmune serum. A and B, Gag expressing insect cells; C and D, non-infected 293 cells; E and F, 293 PERV-PK cells. Magnification, 400×.

FIG. 12. Indirect immunofluorescence analysis of PERV Gag expression using cc-p30D antiserum.

Panels A and C, α-p30D antiserum; panels B and D, preimmune serum. A and B, Gag expressing insect cells; C and D, 293 PERV-PK cells. Magnification, 400×.

FIG. 13. Indirect immunofluorescence analysis of PERV Env expression using α-p15E antiserum.

Panels A, B, D, F, H, α-p15E antiserum; panels C, E, G, preimmune serum. A, Env-A expressing insect cells; B, Env-B expressing insect cells, C, Env-A expressing insect cells; D and E, 293 PERV-PK cells; F and G, 293 cells infected with molecular clone PERV-B(33)/ATG; H. non-infected 293 cells. Magnification, 400×.

FIG. 14. Nucleic acid sequence of clone PK15-PERV-A(58) (SEQ ID NO:1).

FIG. 15. Nucleic acid sequence of clone PK15-PERV-B(213) (SEQ ID NO:2).

FIG. 16. Nucleic acid sequence of clone PERV-A(Bac-130A12) (SEQ ID NO:3).

FIG. 17. Nucleic acid sequence of clone PERV-B (Bac 192B9) (SEQ ID NO:4).

FIG. 18. chromosomal 5′-(FIG. 18A) and 3′-(FIG. 18B) flanking sequence of PK15-PERV-A(58).

FIG. 19. chromosomal 5′-(FIG. 19A) and 3′-(FIG. 19B) flanking sequence of PK15-PERV-B(213).

FIG. 20. chromosomal 5′-(FIG. 20A) and 3′-(FIG. 20B) flanking sequence of PERV-A(Bac130A12).

FIG. 21. chromosomal 5′-(FIG. 21A) and 3′-(FIG. 211B) flanking sequence of clone PERV-B (Bac 192B9).

FIG. 22. chromosomal 3′-flanking sequence of clone PERV-A (Bac463H12)

FIG. 23. chromosomal 3′-flanking sequence of clone PERV-A (Bac-151B10)

The invention is further illustrated by the following non-limiting examples.

### EXAMPLES

### Example 1

Generation and Screening of Porcine and Human λ Bacteriophage Libraries.

A genomic DNA library from PK15 cells was constructed utilizing the lambda FixII/XhoI partial fill-in vector (Stratagene, Amsterdam, The Netherlands) as described previously (Czauderna, F. et al., 2000, J. Virol. 74:4028-4038). The generation of a genomic library from cell line 293 PERV-PK has been reported as well as the screening of bacteriophage libraries with a ³²P-labelled PERV pro/pol probe (Czauderna, F. et al., 2000, J. Virol. 74:4028-4038). Subcloning of DNA inserts from purified λ clones into pBS-KS (Stratagene) was accomplished as described (Czauderna, F. et al., 2000, J. Virol. 74:4028-4038).

### Example 2

Construction of Porcine Genomic BAC Libraries and Preparation of BAC DNA.

The porcine BAC library was constructed from large white pigs using the pBeloBAC11 vector as described previously (Rogel-Gaillard et al., 1999, Cytogenet. Cell Genet. 85:205-211). This genome harbored 20-30 copies of PERV as revealed by Southern blot hybridization (Rogel-Gaillard et al., 1999, Cytogenet. Cell Genet. 85:205-211). Thirty-three BAC clones were mapped by fluorescence in situ hybridization to 22 distinct locations on 14 chromosomes (Rogel-Gaillard et al., 1999, Cytogenet. Cell Genet. 85:205-211). Four of these clones were used in this study and are designated PERV-A(Bac-130A12), PERV-B(Bac192B9), PERV-A(Bac-242D4) and PERV-B(13ac484G4). DNA from individual BAC clones was prepared using conventional alkali lysis of bacteria followed by CsCl gradient centrifugation of the DNA (50000×g overnight). Ethidium bromide was removed from DNA by isobutanol extraction and CsCl was subsequently removed by ethanol precipitation.

### Example 3

Identification of PERV Open Reading Frames.

Isolated PERV sequences were tested for open reading frames (ORF) by means of the protein truncation test (PTT) using the TNT T7 Quick coupled Transcription/Translation System (Promega, Mannheim, Germany) according to the manufacturer's instructions.

Gene sequences for gag, pol and env were amplified in conjunction with a T7 promoter using oligonucleotides T7-PERV-gag-for (CTT GTG CGT CCT TGT CTA CAG, nt 1087-1107), PERV-gag-rev (CTT CAA AGT TAC CCT GGG CTC G; nt 2737-2716), T7-PERV-pol-for (GCT ACA ACC ATT AGG AAA AC, nt 2794-2813), PERV-pol-rev (GAG TTC GGG CTG TCC ACA AGG, nt 6304-6284), T7-PERV-env-for (CCA CTA GAC ATT TGA AGT TC, nt 6116-6136), and PERV-env-rev (GTT AAT AGT TCT AAT CTT AGA AC, nt 8163-8141).

### Example 4

Sequence Analyses of Full Length PERV Sequences (LTRs and Open Reading Frames).

The DNA sequences of both strands of isolated molecular clones were determined by primer walking based on 293-PERV-B(33) (accession no. AJ133816) sequence as described previously (Czauderna, F. et al., 2000, J. Virol. 74:4028-4038) using an ABI 373A or 377 DNA sequencing system (Applied Biosystems, Weiterstadt, Germany) according to the instructions of the manufacturer.

a. Analysis of Open Reading Frames

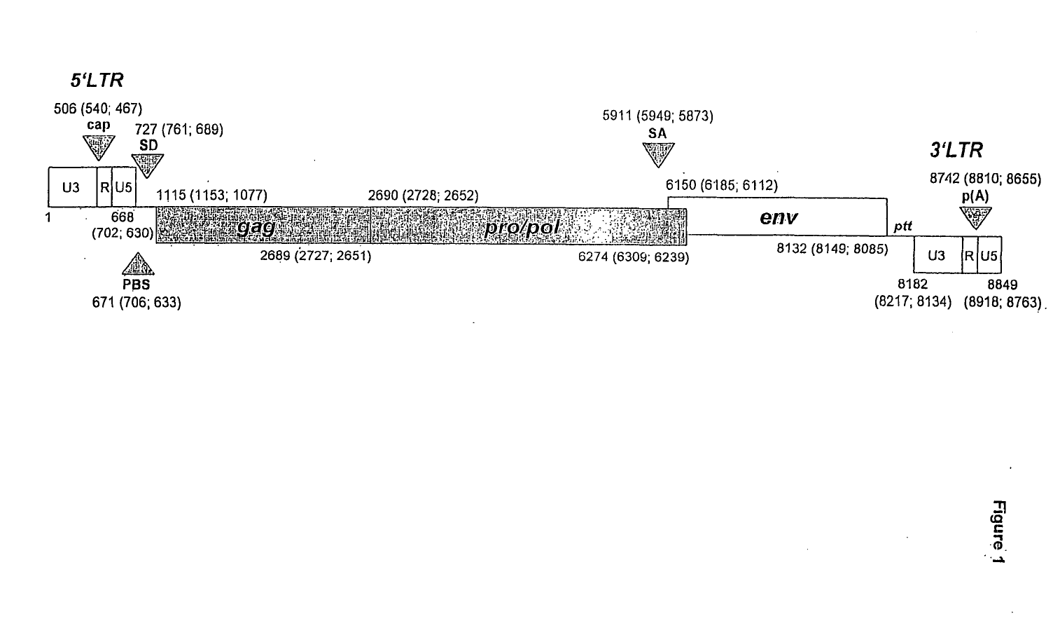

Clone 293-PERV-A(42) derived from human 293 cells and clone PK15-PERV-A(58) isolated from PK15 cells display nucleic acid sequences of 8849 base pairs (bp) and 8918 bp in length, respectively. The gag gene of 293-PERV-A(42) starts at nucleotide (nt) 1115 and is colinear with the pro/pol ORF (nt 2690-6274) (FIG. 1). The amber (UAG) stop codon at nt 2689 separating both genes is suppressed by tRNA_{Gln} as described previously (Akiyoshi, D. E. et al., 1998, J. Virol. 72:4503-4507; Czauderna, F. et al., 2000, J. Virol. 74:4028-4038). The env gene is located at the 3′ end of the proviral sequence (nt 6150-8132) and forms a new reading frame.

PK15-PERV-A(58) shows a similar structure encompassing the genes for gag(nt 1153-2727), pro/pol (nt 2728-6309) and env (nt 6185-8149), respectively. Comparison of the deduced amino acids (aa) of the PERV-A (293-PERV-A(42) and PK15-PERV-A(58) sequences revealed homology scores of 95.8% for Gag, 97.5% for Pro/Pol, and 98.3% for Env compared with 293-PERV-A(42) (Table 1).

PK15-PERV-B(213) displays a sequence of 8763 bp and shows ORF for gag(nt 1077-2651), pro/pol (nt 2652-6239), and env (nt 6112-8085).

The deduced amino acid sequences of PK15-PERV-B(213) show high homology scores compared to 293-PERV-A(42) for Gag (99.6%) and Pro/Pol (98.9%), respectively (Table 1). The comparison of Env sequences of PK15-PERV-B(213) and 293-PERV-A(42) shows 68.0% homology to each other. A comparison of the amino acid sequence of PK15-PERV-B(213) and the previously characterized 293-PERV-B(43) (Czaudema F. et al., 2000, J. Virol. 74:4028-4038) revealed high homology scores for Gag (99.4%), Pro/Pol (99.3%) and Env (99.1%).

PK15-PERV-B(213) harbors the longest pro/pol gene. The gene bears one additional codon (nt 62346237, coding for glutamine) compared to pro/pol of 293-PERV-A(42) and PK15PERV-A(58) and another additional codon (nt 5951-5953, coding for arginine) compared to PK15-PERV-A(58). Likewise, in the pro/pol of 293-PERV-A(42) an additional arginine (nt 5989-5991) is found compared to PK15-PERV-A(58). Thus PK15-PERV-A(58) bears the shortest pro/pol gene.

The env gene of PK15-PERV-A(58) demonstrates a curtailment of 18 nt compared to 293PERV-A(42) (nt 8115-8132) at the 3′-end of the sequence. The specific differences of PERV-A and PERV-B env are also reflected by the 9 nt difference in length between the sequences of PK15-PERV-B(213) and 293-PERV-A(42) env (1973 nt and 1982 nt, respectively).

The homology data are summarized in Table 1.

TABLE 1
Comparison of nucleotide and amino acid sequences of
293-PERV-A(42) gag, pro/pol and env ORF with
PK15-PERV-A(58) and PK15-PERV-B(213)

| | Percent nucleotide homology | | |
|---|---|---|---|
| | and amino acid homology (in brackets) | | |
| | with 293-PERV-A(42) gene (protein) | | |
| Virus | Gag | pro/pol | Env |
| PK15-PERV-A(58) | 95.4 (95.8) | 97.2 (97.5) | 98.1 (98.3) |
| PK15-PERV-B(213) | 99.9 (99.6) | 99.3 (98.9) | 73.9 (68.0) |

Homology scores were revealed using sequence analysis software DNASIS (Hitachi).

Furthermore, the above-mentioned proviral PERV clones demonstrate highly conserved amino acid motifs of mammalian type C retroviruses (Akiyoshi, D. E. et al., 1998, J. Virol. 72:4503-4507; Czauderna, F. et al., 2000, J. Virol. 74:4028-4038) as summarized in Table 2.

TABLE 2
Highly conserved amino acid motifs of mammalian type C retroviruses present in
293-PERV-A(42), PK15-PERV-A(58) and PK15-PERV-B(213)

| Protein | Consensus sequence | PERV sequence | Nucleotide position |
|---|---|---|---|
| N-terminus of | Asn₋₁-Met₁-Gly₂-Gln₃-Thr₄¹ | Identical | 1115-1127; 1153-1165; |
| Gag | | | 1077-1089 |
| N-terminus of | Proline² | Identical | 1697-1699; 1735-1737; |
| p30 | | | 1659-1661 |
| C-terminus of | Thr-Lys-X-Leu | Thr-Lys-Ile-Leu³ | 2463-2475; 2501-2513; |
| p30 | | | 2425-2437 |
| Cys-His box in | Cys-Xaa2-Cys-Xaa4-His-Xaa4- | Identical | 2592-2634; 2630-2672; |
| p10 | Cys⁴ | | 2554-2596 |
| Aspartyl | Leu-Leu/Val-Asp-Thr-Gly-Ala- | Leu-Val-Asp-Thr-Gly-Ala- | 2762-2785; 2724-2747; |
| protease | Asp-Lys⁵ | Glu/Lys-His⁶ | 2800-2823 |
| RNA-dependent | Tyr-X-Asp-Asp (YXDD)⁷ | Tyr-Val-Aap-Asp (YVDD) | 3557-3569; 3597-3609; |
| polymerase | | | 3521-3533 |
| (RT) | | | |
| Cleavage site | Arg/Lys-X-Lys-Arg⁸ | Arg-Pro-Lys-Arg | 7525-7537; 7560-7572; |
| gp70/p15E | | | 7478-7490 |

Motifs of retroviral consensus sequences (nt positions) are given for 293-PERV-A(42), PK15-PERV-A(58), and PERV-B(213).
Foot notes
¹motif in MoMLV (Shinnick, T. M. et al., 1981, Nature 293: 543-548) and PERV-MSL (Akiyoshi, D. E. et al., 1998, J. Virol. 72: 4503-4507);
²(Oroszlan, S. et al., 1981, Virology 115: 262-271);
³identical in BaEV (acc. no.: D10032), GaLV (acc. no.: NC_001885) and KoRV (AF151794);
⁴(Green, L. M. and J. M. Berg. 1989, Proc. Natl. Acad. Sci. USA 86: 4047-4051, 24);
⁵(Coffin, J. 1996. Retroviridae: the viruses and their replication, p. 1767-1847. In B. N. Fields, D. M. Knipe, P. M. Howley, et al. (ed), Fields virology, 3^{rd} ed. Lippincott-Raven Publishers, Hagerstown, Md.);
⁶in PK15-PERV-A(58), 7^{th} amino acid is lysine;
⁷(Xiong, Y. and T. H. Eickbush. 1990, EMBO J. 9: 3353-3362);
⁸(Akiyoshi, D. E. et al., 1998, J. Virol. 72: 4503-4507; LeTissier, P. et al., 1997, Nature 389: 681-682).

The sequences of clones PERV-A(Bac-130A12) (SEQ ID NO:3) and PERV-B(Bac-192B9) (SEQ ID NO:4) were determined displaying proviruses of 8918 bp and 8840 bp, respectively.

While the sequence of the LTRs and viral genes were determined separately, they were assembled for this analysis. The gag gene of clone PERV-A(Bac-130A12) ranges from nt 1153 to nt 2727 and the pro/pol ORF is located in the same reading frame (nt 2728-6309). The env gene forms the third ORF (nt 6185-8149). Clone PERV-A(Bac-130A12) has been chromosomally assigned and maps to 1q2.4 (Rogel-Gaillard et al., 1999, Cytogenet. Cell Geizet. 85:205-211).

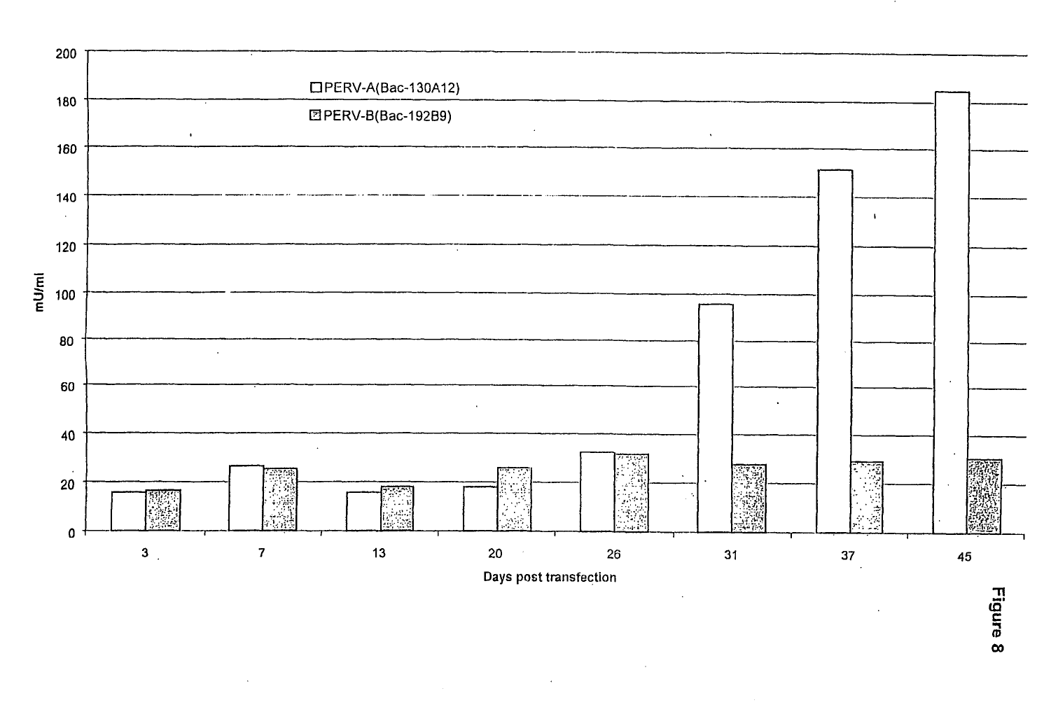

PERV-B(Bac-192B9) shows a similar structure and harbors gag (nt 1115-2689), pro/pol (nt 2837-6277) and env (nt 8173-8123) genes, respectively. However, two stop codons at nt 4687 and nt 5251 within the pro/pol sequence disrupt the open reading frame (ORF) and, as a consequence, prevent this clone from replication (FIG. 8). The chromosomal location of PERV-B(Bac-192B9) is 7p1.1, and therefore maps to the SLA.

Sequences of PERV-A(Bac-130A12) and PERV-B(Bac-192B9) showed close relationship to proviral PERV sequences described previously (Czaudema et al., 2000). PERV-A(13ac130A12) is almost identical to PK15-PERV-A(58) demonstrating homologies of approximately 99% for the LTRs and the viral genes. However, both clones appear to map to different chromosomal locations as deduced from the flanking sequences. PERV-A(Bac130A12), in comparison to 293-PERV-A(42) (Czauderna F. et al., 2000, J. Virol. 74:4028-4038), shows slightly lower homologies of approximately 95% within the retroviral genes and a completely different LTR structure. PERV-B(Bac-192B9) demonstrates high homology (approximately 98% to clone 293-PERV-B(33) (Czaudema F. et al., 2000, J. Virol. 74:4028-4038), however, the LTR of this provirus is similar to that of class A clone 293-PERV-A(42) which bears a characteristic 39-bp repeat structure in U3 (Czauderna F. et al., 2000, J. Virol. 74:4028-4038).

The homology data for SEQ ID NO:4 and SEQ ID NO:5 are summarized in Table 3.

TABLE 3
Comparison of nucleotide and amino acid sequences of
PERV-A(Bac-130A12) and PERV-B(Bac-192B9) gag, pro/pol
and env ORF with other proviral PERV sequences

| | Percent nucleotide homology and amino | | |
|---|---|---|---|
| | acid homology (in brackets) with appropriate | | |
| | PERV sequence | | |
| | PERV-A(Bac- | | PERV- |
| | 130A12) compared | PERV-A(Bac- | B(Bac-192B9) |
| | with PK15-PERV- | 130A12) compared | with PERV-B(33)/ |
| Gene | A(58) | with PERV-A(42) | ATG |
| LTR | 99.9% | 63.6% | 99.4%* |
| gag | 99.8% (98.9%) | 95.4% (95.8%) | 98.7% (98.7%) |
| pro/pol | 99.7% (98.4%) | 96.9% (96.6%) | 98.7% (—) |
| env | 99.8% (98.0%) | 97.5% (96.3%) | 99.1% (98.9%) |

*compared to the LTR of PERV-A(42)
Homology scores were revealed using sequence analysis software DNASIS (Hitachi).

b. Analysis of LTR Sequences.

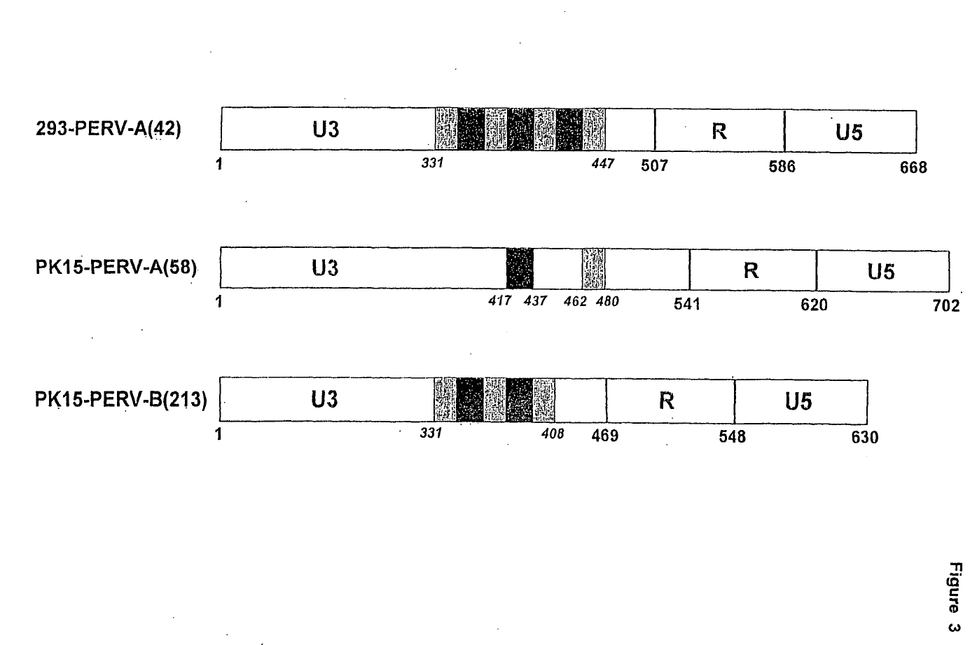

The long terminal repeats CLTR) of PK15-PERV-A(58) and PK15-PERV-B(213) (FIG. 3) exhibit major differences. The LTR of these proviral PERV are limited by the inverted repeat sequence TGAAAGG/CCTTTCA, as described for the previously characterized clones 293PERV-B(33) and 293-PERV-B(43) (Czauderna F. et al., 2000, J. Virol. 74:4028-4038) Furthermore, a box of 39-bp repeats is found in the U3 region of 293-PERV-A(42) and PK15PERV-B(213), each repeat consisting of subrepeats of 21 bp and 18 bp motifs. For 293PERV-A(42), three consecutive repeats ranging from nt 331 to nt 447 are found. The LTR of PK15-PERV-B(213) exhibits two repeats (nt 331408). In both LTRs, an 18-bp repeat is found preceding the triplex and duplex repeat box, respectively. Thus, the LTR of PK15WO PERV-B(213) resembles the LTR of molecular clone 293-PERV-B(43) (Czaudema, F. et al., 2000, J. Virol. 74:4028-4038.), showing a homology of 99.0%.

The LTR of PK15-PERV-A(58) harbors one 21-bp and one 18-bp subrepeat, both showing two nt exchanges, which are separated from each other (nt 417437 and nt 462-480, respectively) (FIG. 3). The U3 sequence of PK15-PERV-A(58) shows homologies of 59.0% and 65.2% compared to the LTR of 293-PERV-A(42) and PK15-PERV-B(213), respectively.

In contrast, the R and U5 sequences of PK15-PERV-A(58) demonstrate homologies of 97.5% for 293-PERV-A(42) and 88.0% for PK15-PERV-B(213).

### Example 5

Phylogenetic Relationship of PERV Clones.

A comparison of the proteins of different PERV, including PERV-MSL (Akiyoshi, D. E. et al., 1998, J. Virol. 72:4503-4507), clones 293-PERV-B(33)/ATG and 293-PERV-B(43) (Czaudema, F. et al., 2000, J. Virol. 74:4028-4038), as well as the clones 293-PERV-A(42), PK15-PERV-A(58) and PK15-PERV-B(213) described here, revealed different assignments of individual clones by phylogenetic analysis wig. 2).

Phylograms are based on full-length open reading frames for Gag (A), Pro/Pol (13), and Env (C) (see also Table 1). Relative distances are indicated by scale bars (0,1 indicates 10% divergence). Phylograms were generated using Phylip 3.574c and the Prodist and Neighbor programs (http:H/evolution.genetics.washington.edu/phylip.html).

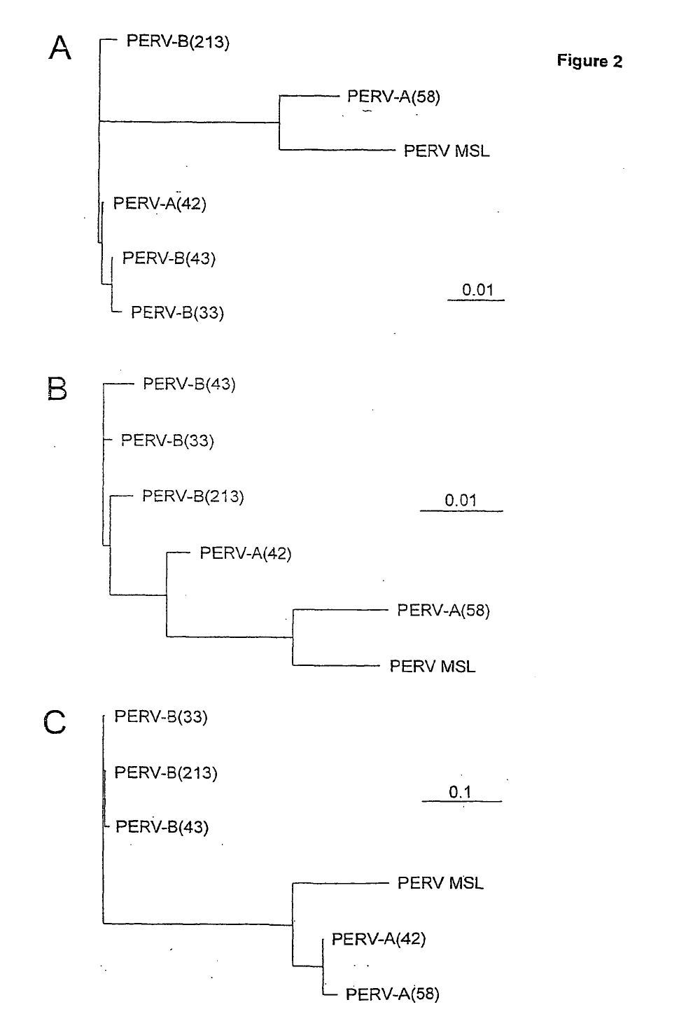

For Gag, a clustering of the clones derived from human 293 cells was revealed, whereas Gag of PK15-PERV-A(58) is closer related to Gag of PERV-MSL than to Gag of PK15-PERV-B(213) (FIG. 2A). Thus, it appears that the selection achieved by serial passages of PERV on human cells (Czauderna, F. et al., 2000, J. Virol. 74:4028-4038; Patience, C. et al., 1997, Nat. Med. 3:282-286) has favored a certain type of Gag (FIG. 2A). The Pro/Pol sequences demonstrate a distribution according to the appropriate class of PERV (FIG. 2B). In regard of the class-specific assignment, particularly for the class B clones, it could be speculated based on Pro/Pol sequences that the different PERV-B clones have arisen from one ancestral provirus (FIG. 2B). The “native” clone PK15-PERV-B(213) is closely related to 293-derived clones 293-PERV-B(33) and 293-PERV-B(43). However, the two PERV-A clones show a higher level of divergence for Pro/Pol. Env shows a class-like distribution (LeTissier, P. et al., 1997, Nature 389:681-682) as expected where the class B sequences form one branch (FIG. 2C). Interestingly, clones 293-PERV-A(42) and PK15-PERV-A(58) are located proximal to PERV MSL in Env. PERV MSL demonstrates general proximity to PK15-PERV-A(58) for all three ORF.

### Example 6

Detection of Proviral PERV-Integration.

In order to detect proviral PERV integration, genomic DNA was isolated from different cell lines grown to confluence by standard procedures (Sambrook, J., E. F. Fritsch, and T.

Maniatis. 1989. Molecular cloning: a laboratory manual, 2^{nd} ed. Cold Spring Harbor Laboratory Press, Cold Spring Harbor, N.Y.). The genomic DNA was subsequently analyzed via PCR for the presence of proviral integrations.

For analysis via PCR, proviral integration of PERV was tested by amplification of pro/pol sequences using oligonucleotides PK1 (5′-TTG ACT TGG CAG TGG GAC GGG TAA C-3′, nucleotide (nt) 2886-2910) and PK6 (5′-GAG GGT CAC CTG AGG GTG TTG GAT-3′, nt 3700-3677) in a first amplification and PK2 (5′-GGT AAC CCA CTC GTT TCT GGT CA3′, nt 2905-2927) and PK5 (5′-CTG-TGT AGG GCT TCG TCA AAG ATG-3′, nt 3657-3634) in a nested amplification. Nt positions refer to 293-PERV-A(42).

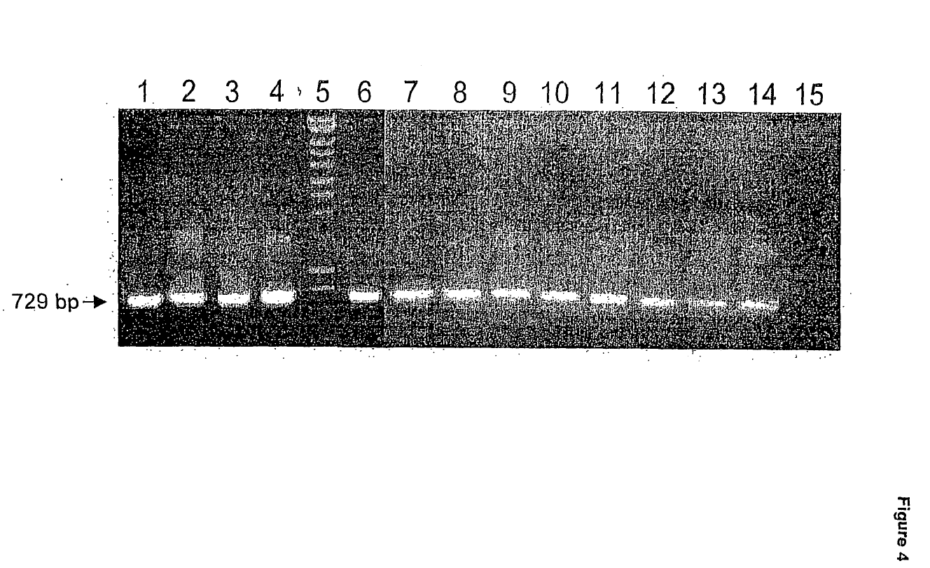

All cell lines used in infection studies, 293, HeLa, D17, and PG4, showed the expected 729 bp amplification product after infection as revealed for 293-PERV-A(42), PK15-PERV-A(58), and PK15-PERV-B(213) (FIG. 4). The existence of episomal PERV DNA was excluded by using cesium chloride gradient purified genomic DNA from infected cell lines for amplification of the 729 bp pro/pol fragment.

### Example 7

Detection of Productive Infectivity of Different PERV Clones by Indirect Immunofluorescence Analysis.

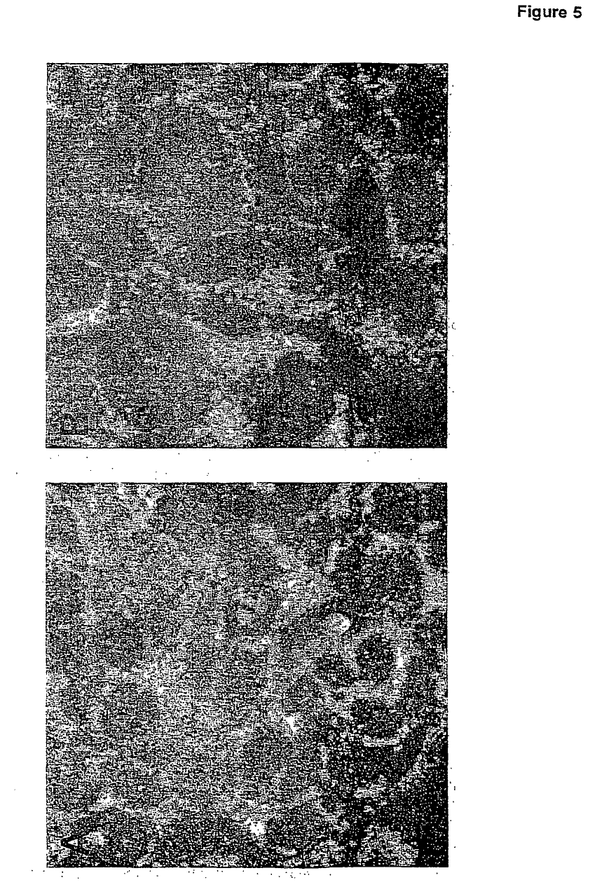

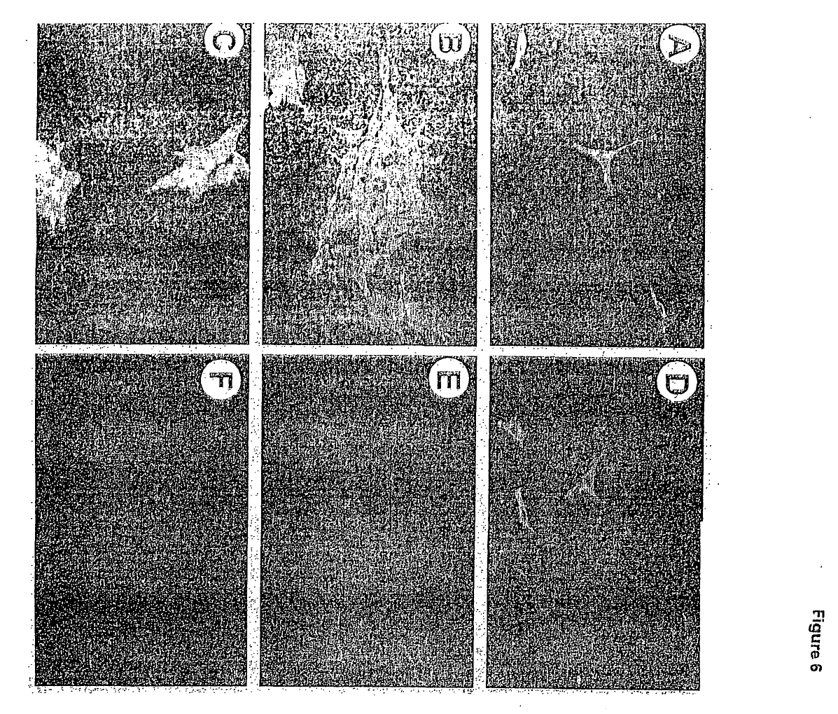

As different cell lines have been described to be susceptible for PERV infection (Takeuchi, Y. et al., 1998, J. Virol. 72:9986-9991), the ability of 293-PERV-A(42), PK15-PERV-A(58), and PK15-PERV-B(213) to productively infect cells was investigated by indirect immunofluorescence analyses using a PERV-specific Gag p10 antiserum (Krach, U. et al., 2000, Xenotransplantation 7:221-229). Human 293 and HeLa cells as well as canine D17 cells and feline PGA cells were infected with PERV and fixed 48 to 72 h post infection (p.i.) with 2% formaldehyde. Indirect immunofluorescence analyses were performed as described previously (Krach, U. et al., 2000, Xenotransplantation 7:221-229). Distinct signals were obtained for all three viruses after incubation with the antibody (FIG. 5). 293-PERV-A(42) and PK15-PERV-B(58) showed significant Gag expression 8-12 days post infection in all cell lines similar to the pattern found for 293 cells infected with molecular clone 293-PERV-B(33)/ATG (Krach, U. et al., 2000, Xenotransplantation 7:221-229; data not shown). PK15PERV-B(213), however, showed a lower degree of Gag expression (data not shown).

### Example 8

Detection of the Presence of Infectious and Replication-Competent Viral Particles by RT Analysis.

To confirm the presence of infectious and replication-competent viral particles, RT activities in the supernatant of cell lines were determined in the course of infection with PERV. Membrane filtered cell-free supernatants were tested for RT-activity employing the C-type RT activity assay (Cavidi Tech Ab, Uppsala, Sweden) according to the manufacturers instructions (protocols B).

Infectivity was tested by inoculation of semi confluent cultures of susceptible cell lines with cell-free supernatants of producer cells after filtration through 0.45 μm pore size membranes (Sartorius, Göttingen, Germany). Cell-free supernatants from D17, PG4, HeLa and 293 cells infected with the molecular clones 293-PERV-A(42), PK15-PERV-A(58), and PK15-PERV-B(213) were collected up to 51 days post infection (p.i.).

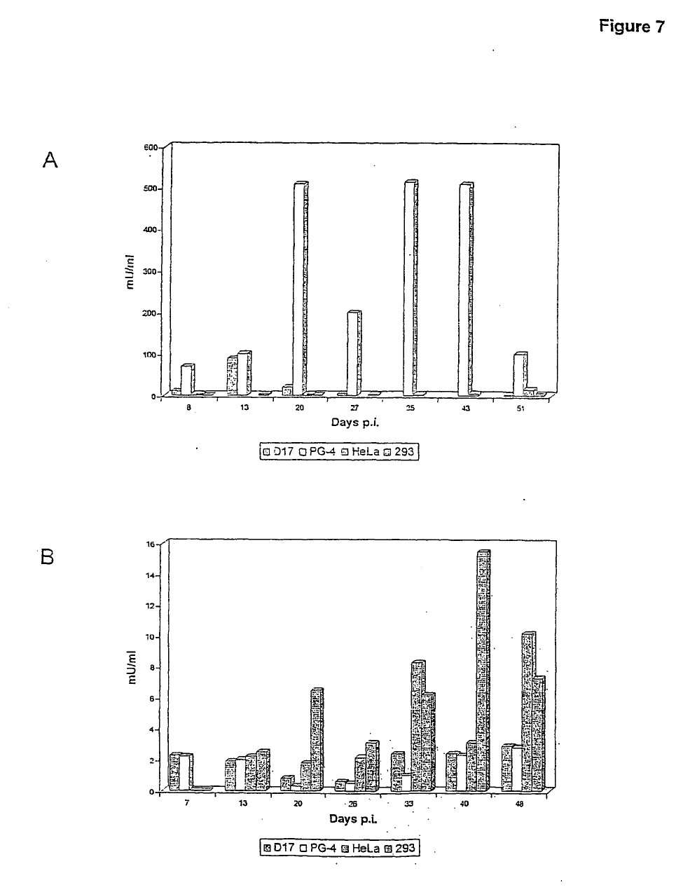

In the case of clone 293-PERV-A(42), RT activity of up to 500 mU/ml was found for PG4 cells (FIG. 7A). Furthermore, 293-PERV-A(42) initially demonstrated an activity of 100 mU/ml (day 13) after infection of D17 cells which declined from day 20 on. 293-PERV-A(42) demonstrated only weak RT activity on HeLa cells at day 51 and did not replicate on 293 cells. Clone PK15-PERV-A(58) demonstrated RT activities barely above background (FIG. 7B). In contrast to clone 293-PERV-A(42), clone PK15-PERV-A(58) showed RT activity on 293 cells at day 40 p.i. PK15-PERV-B(213) demonstrated RT activities upon infection of 293 and HeLa cells (data not shown). For 293 cells, a transient activity of up to 4 mU/ml was detected at day 21. HeLa cells showed RT activities ranging from 2 to 4 mU/ml until day 57. All other cell lines revealed only background activities (data not shown).

### Example 9

Generation of PERV flanking sequences for screening of proviral integration sites[RRT2]. Chromosomal sequences adjacent to the proviral sequences of clones PERV-A(Bac-130A12), PERV-B(Bac-192B9) were revealed by inverse PCR, using approaches essentially as described earlier (Tönjes et al., 1999, J. Virol. 73:9187-9195). Amplification products were cloned into pGEM-T Easy and sequences were determined. Restriction enzymes and oligonucleotide primers used for appropriate inverse PCR reactions are given in Table 4.

TABLE 4

Sequences and positions of oligonucleotide primers
used for inverse PCR to generate adjacent
chromosomal sequences of clones PERV-A(Bac-130A12)
and PERV-B(Bac-192B9).

| | |
|---|---|
| PERV-A(Bac-130A12) | PERV-B(Bac-192B9). |

| | 5′- | 3′- | 5′- | 3′- |
|---|---|---|---|---|
| | flanking | flanking | flanking | flanking |
| | sequence | sequence | sequence | sequence |
| Restriction | EcoR V | Kpn I | EcoR V | Afl II |
| enzyme | | | | |
| Forward | PK27 | PK22 | PK27 | PK22 |
| primer | | | | |
| Reverse | PK26 | PK21 | PK26 | PK21 |
| primer | | | | |
| Forward | A13 | — | A13 | PK30 |
| primer | | | | |
| (nested | | | | |
| PCR) | | | | |
| reverse | PK15 | — | PK15 | A19 |
| primer | | | | |
| (nested | | | | |
| PCR) | | | | |

| Oligonucleotide | |
|---|---|
| primer | Primer sequence |
| PK15 | ACAGACACTCAGA |
| | ACAGAGACGCC |
| PK21 | AAGGACCACTTCCT |
| | CAGGATGGTA |
| PK22 | AAAGAGAACCCGT |
| | ATCCCTTACCC |
| PK26 | ACGCACAAGACAA |
| | AGACACACGAA |
| PK27 | CTTGTCTACAGTTT |
| | TAATATGGGA |
| PK30 | TGGATGACCACCCT |
| | GCTTTCTGCT |
| A13 | CGGTATTTTCTTGA |
| | GAGGCTC |
| A19 | ACAGTGACACCCG |
| | TATCAGG |

### Example 10

Differentiation of PERV Classes by PCR.

To distinguish PERV-A and PERV-B proviral sequences, env-A and env-B specific oligonucleotide primers were employed in PCR experiments. Oligonucleotides used are env-A-for (CAA TCC TAC CAG TTA TAA TCA ATT, nt 6638-6661), env-A-rev (TCG ATT AAA GGC TTC AGT GTG GTT, nt 7334-7311), env-B-for (GTG GAT AAA TGG TAT GAG CTG GGG, nt 6711-6734), and env-B-rev (CTG CTC ATA AAC CAC AGT ACT ATA, nt 7287-7264). Nt positions for env-A and env-B refer to 293-PERV-A(42) and PK15PERV-B(213), respectively.

Nucleotide sequence accession numbers. Sequences used for homology analyses are 293PERV-B(33) (AJ133816), 2⁹3-PERV-B(43) (AJ133818), and PERV-MSL (AF038600).

### Example 11

Generation and Testing of PERV Antibodies

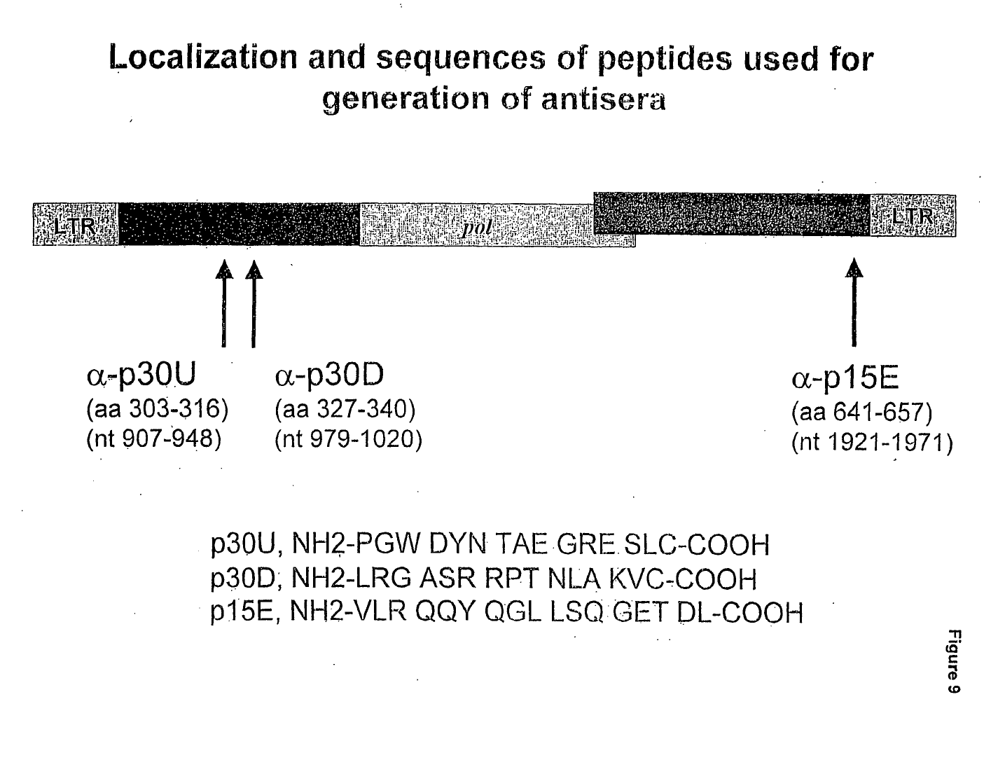

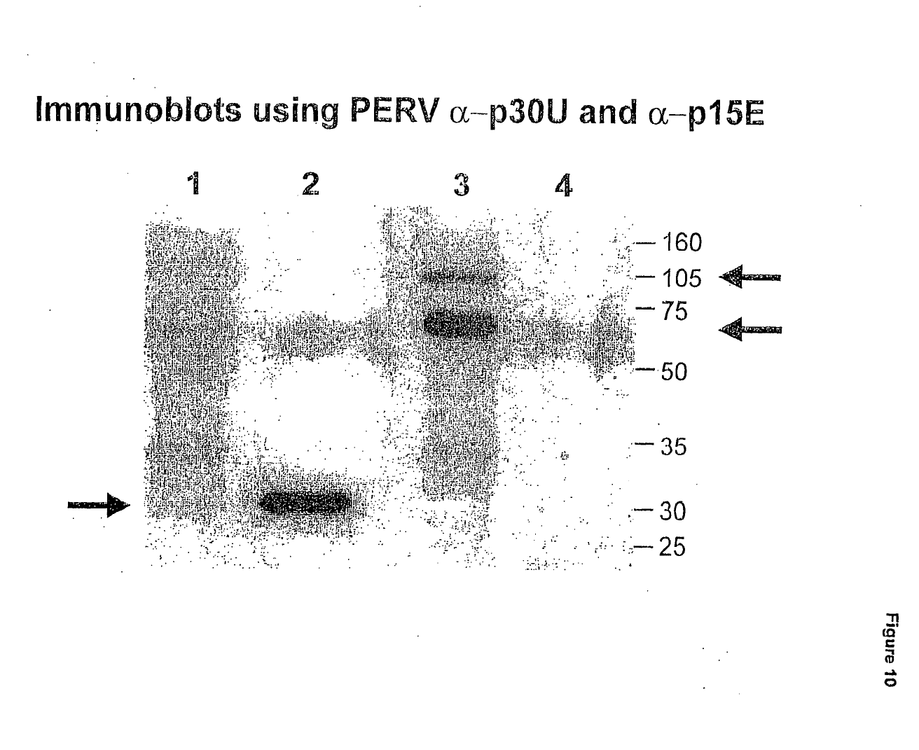

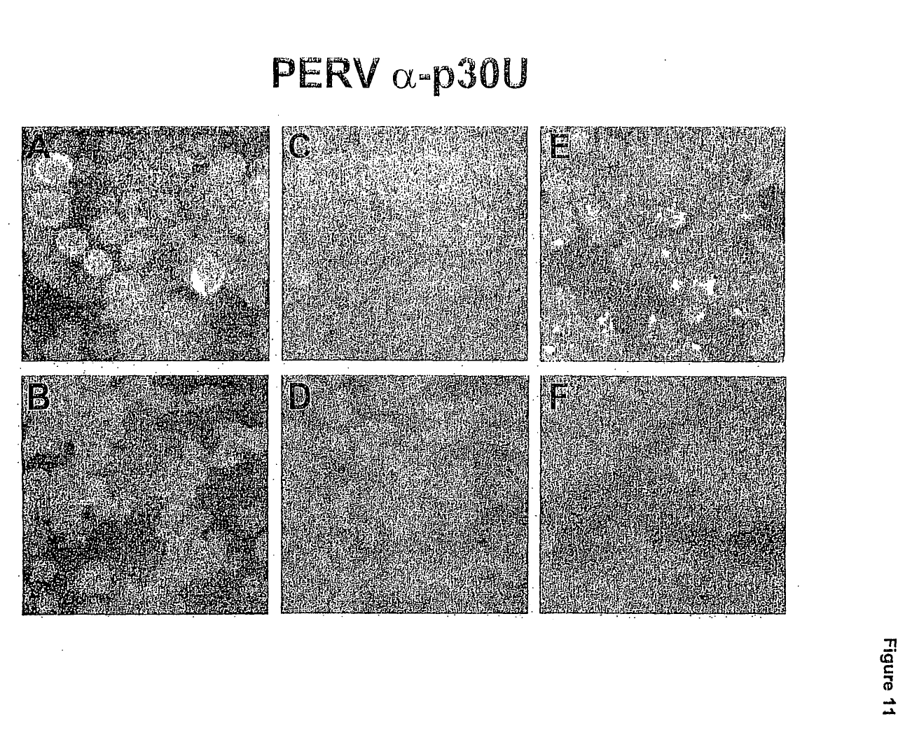

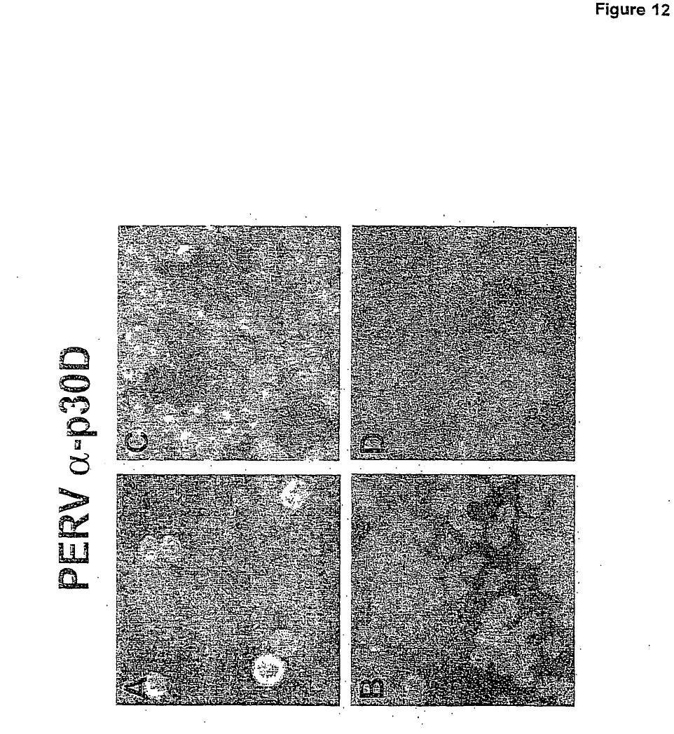

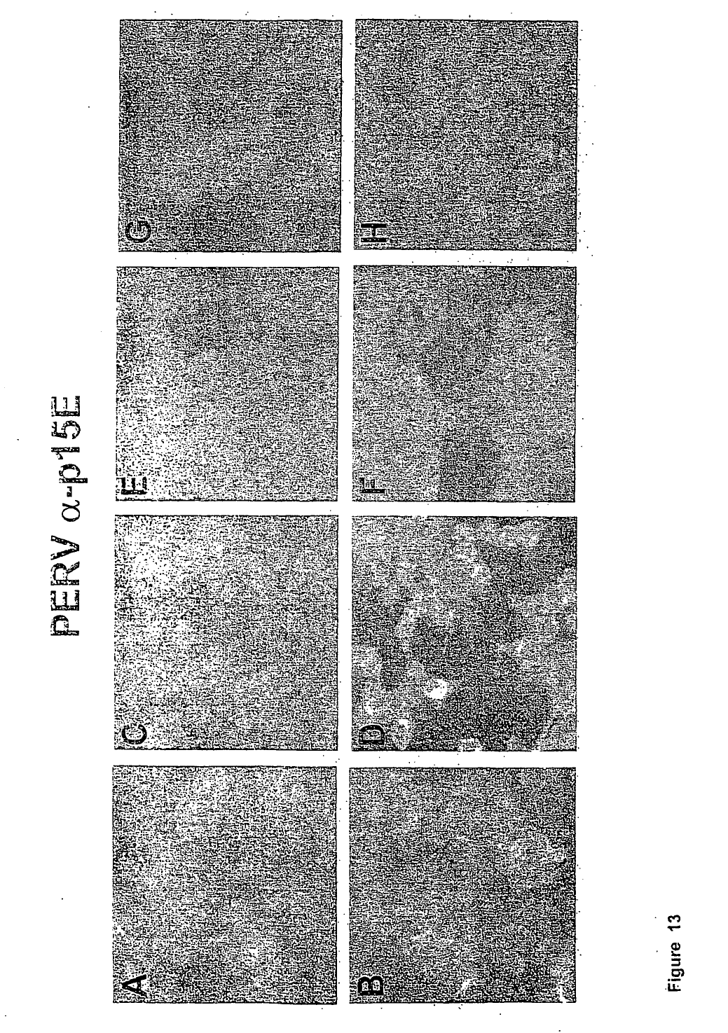

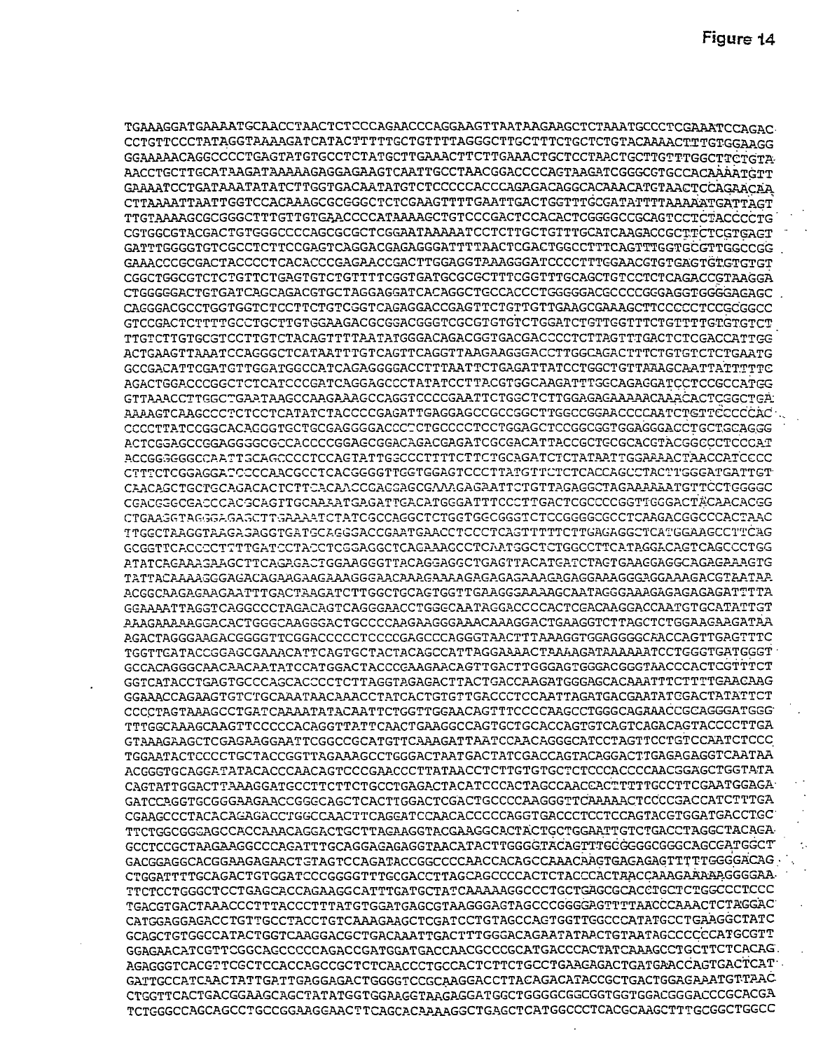

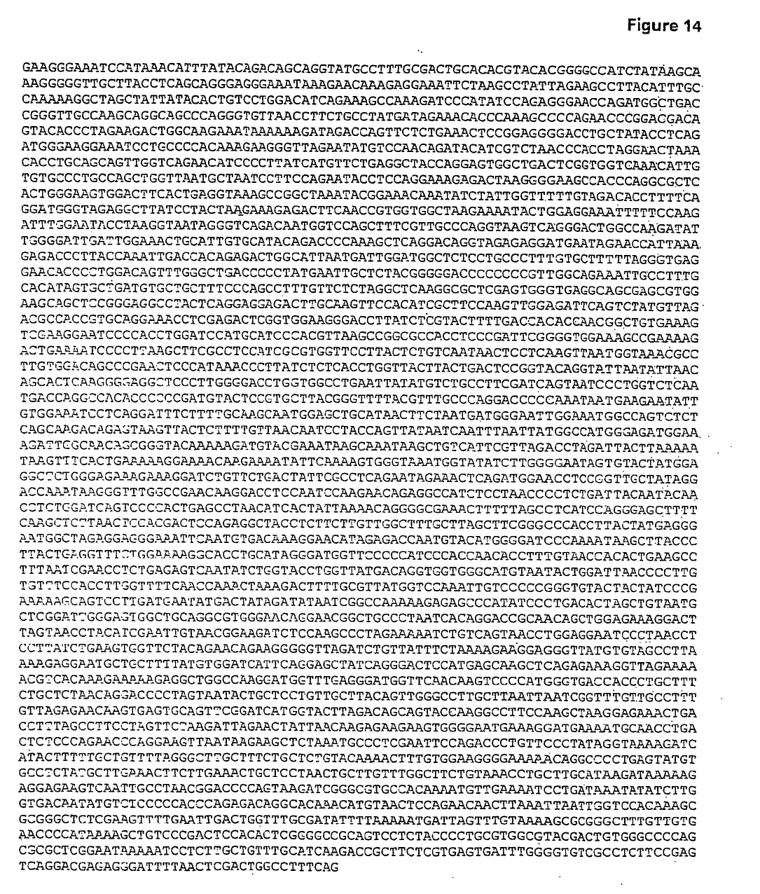

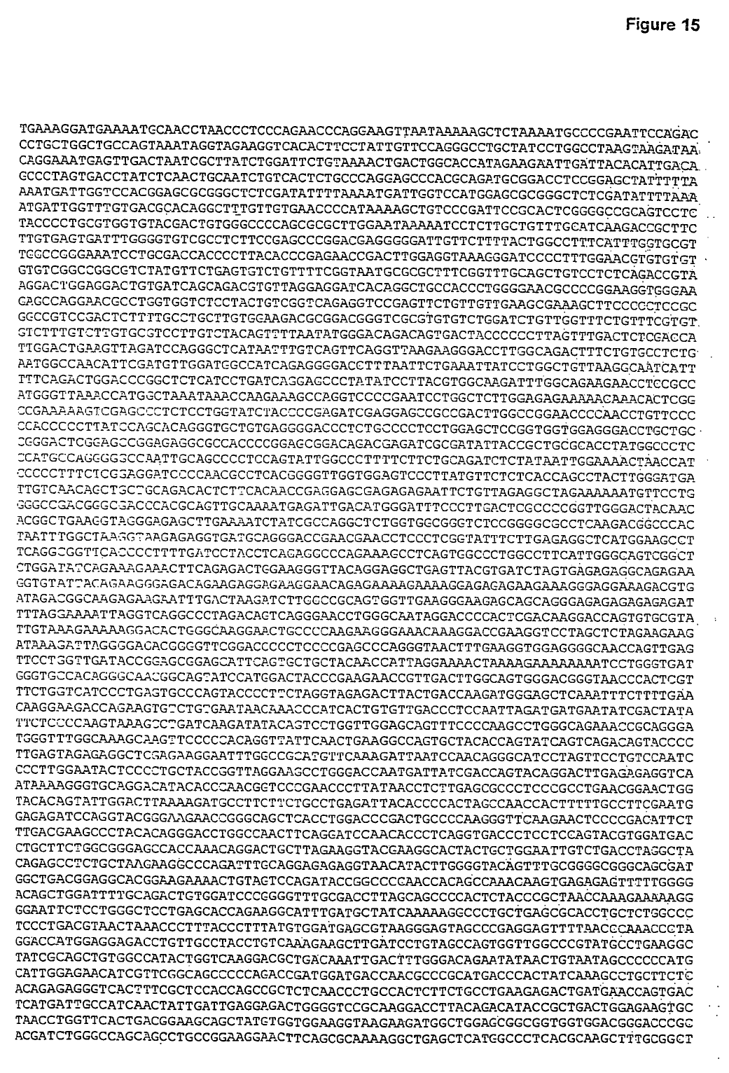

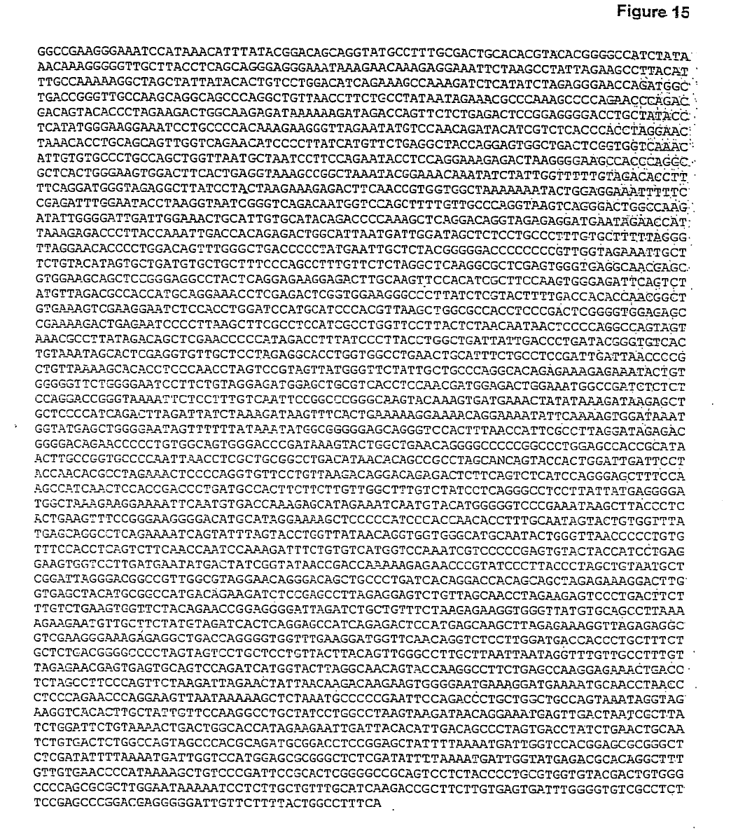

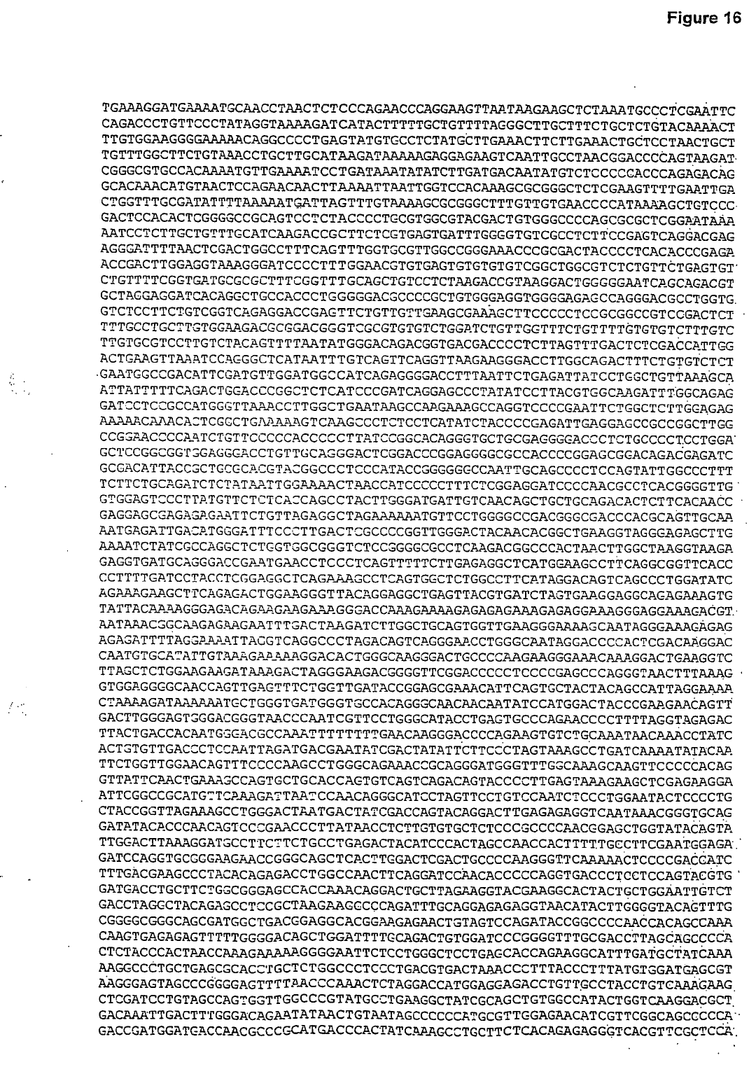

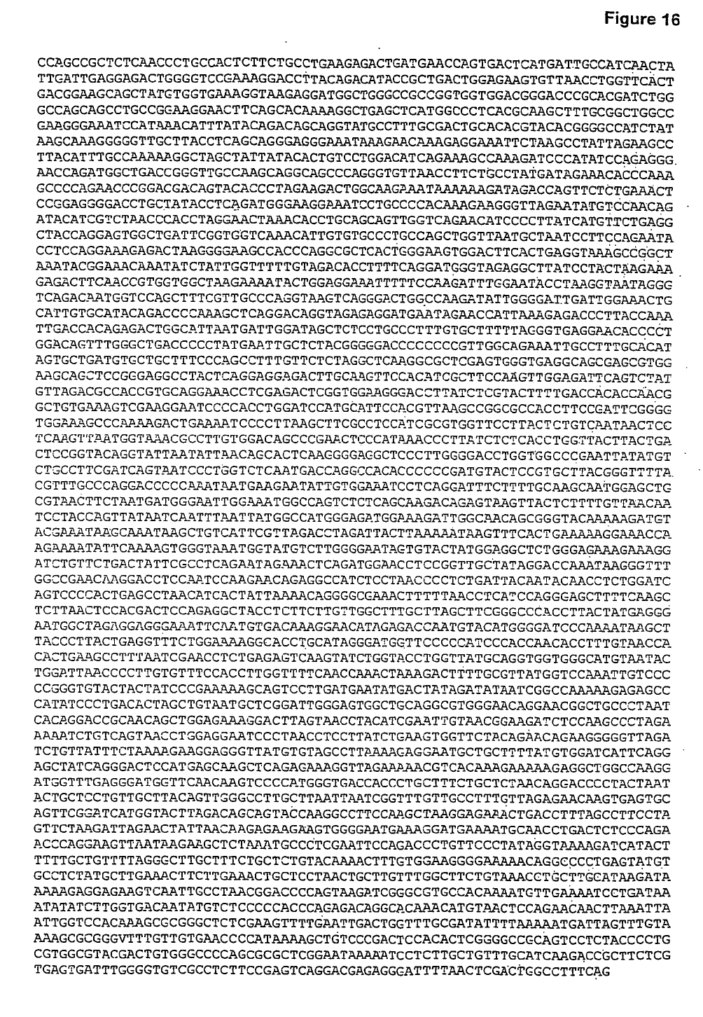

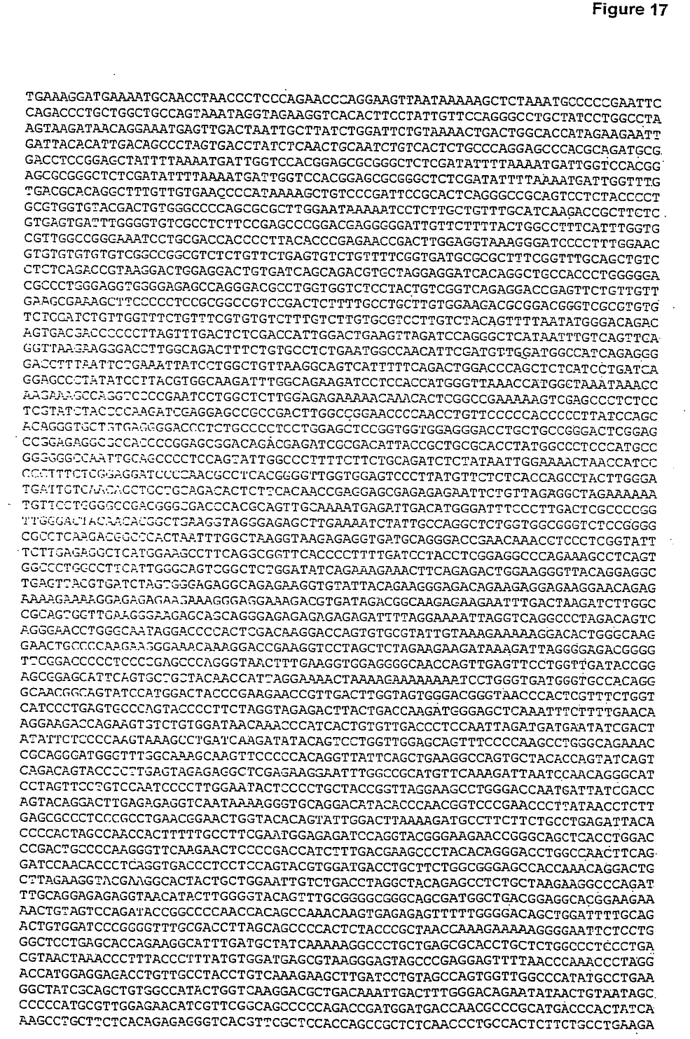

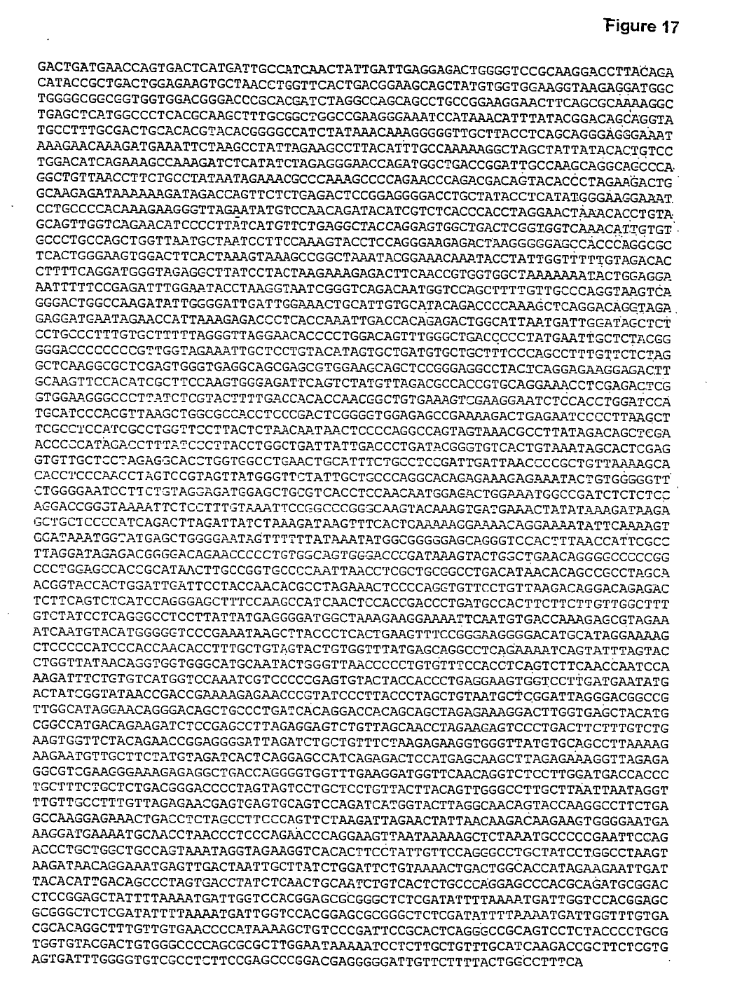

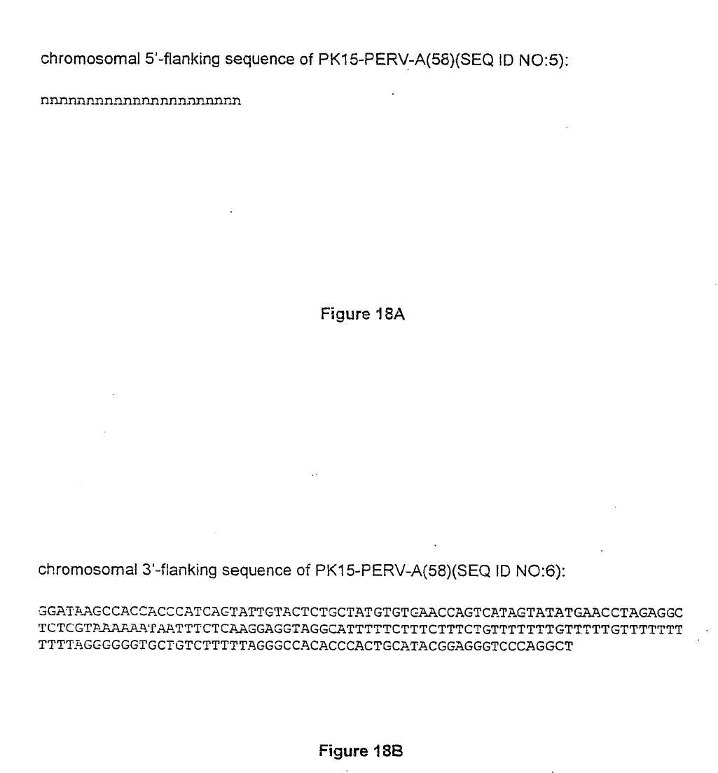

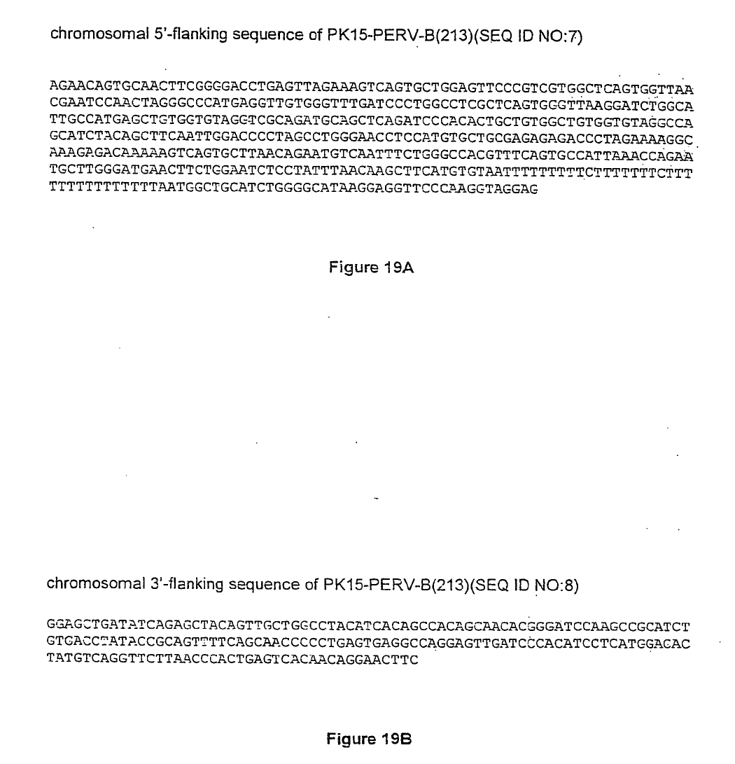

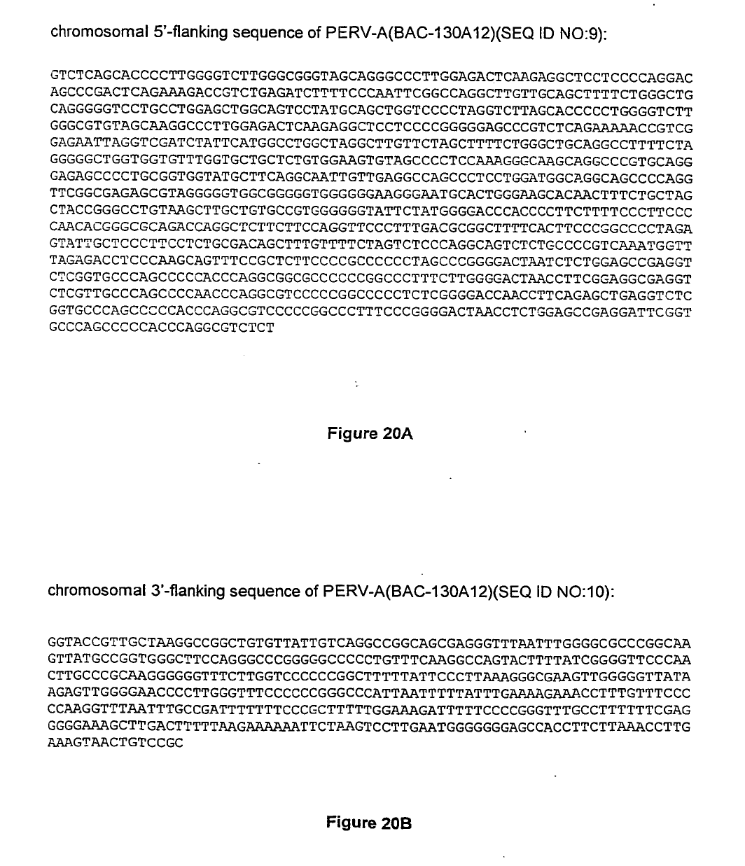

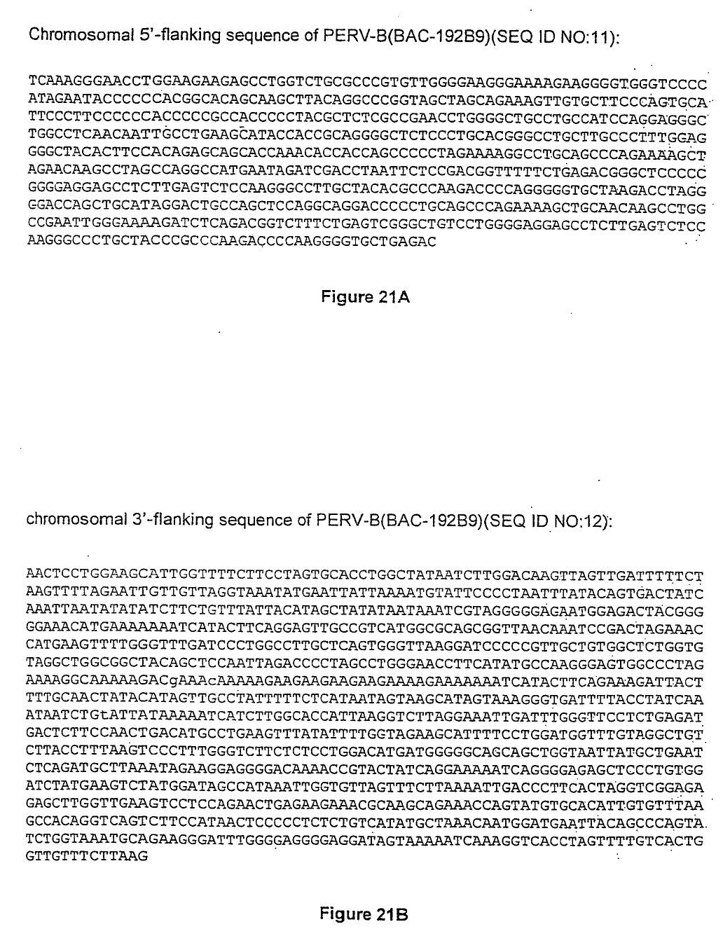

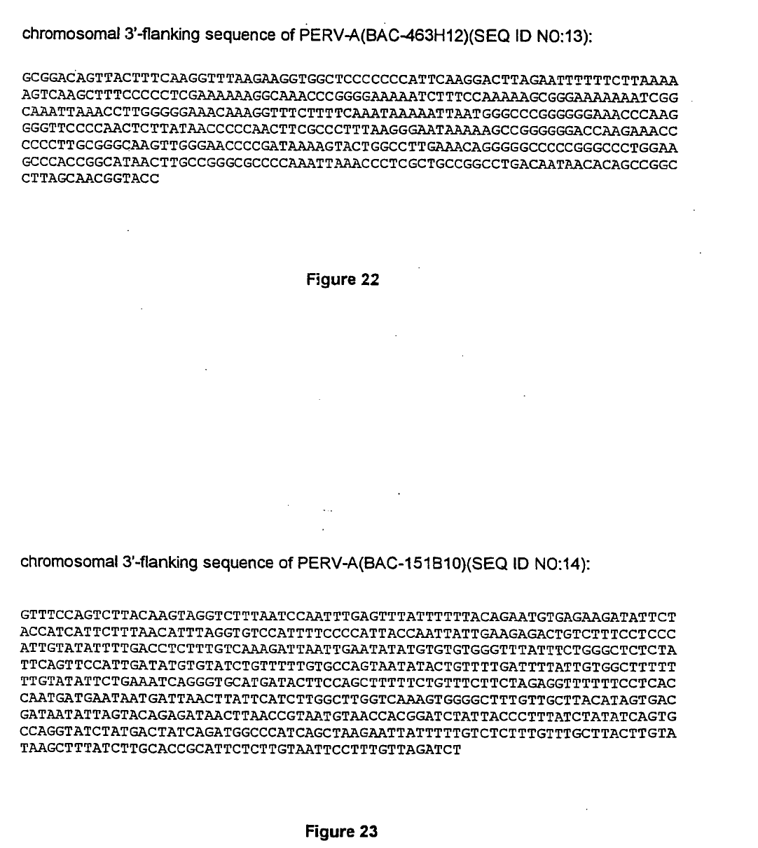

a. Generation of PERV antisera. The peptides p30U (NH2—PGW DYN TAE GRE SLCCOOH, amino acid (aa) 303-316, nucleotide (nt) 907-948), p30D (NH2-LRG ASR RPT NLA KVC-COOFL aa 327-340, nt 979-1020), and p15E (H2-VLR QQY QGL LSQ GET DL-COOH, aa 641-657, nt 1921-1971) derived from the Gag and Env sequences of PERV were used to raise antisera (FIG. 9). Positions refer to clone PERV-B(33)/ATG (Czauderna et al., 2000).

The antigens were commercially synthesized by Eurogentec (Belgium), purified by HPLC, and linked to keyhole limpet hemocyanin (KLH) for immunizations. Polyclonal antisera were generated in rabbits using either complete Freund's adjuvant in case of the initial immunization or incomplete Freund's adjuvant in case of the boost immunizations.

b. Cells. 293 human embryonic kidney cells (ECACC, no. 85120602) and 293 cells that constitutively produce PERV (293 PERV-PK) were kindly provided by Dr. Weiss, London. In addition, 293 cells infected with molecular clone PERV-B(33)/ATG which produced infectious virions were used (Czauderna et al., 2000). SHi5 insect cells (Hi5 cells adapted to growth in serum-free media) have been described previously (Krach et al., 2000). Expression of PERV Gag and Env proteins was achieved by infection of Shi5 cells with recombinant baculoviruses Bac-PERV-G, Bac-PERV-E(A) or Bac-PERV-E(B) bearing the PERV gag (nt 1145-2728), env-A (nt 6153-8114) or env-B (nt 6183-6208) genes, respectively, and subsequent immunofluorescence studies. The expressed sequences were derived from clones PERV-A(42) [env-A] and PERV-B(33) [gag, env-B] (Czaudema et al., 2000) and cloned into baculovirus transfer vector pBac2 cp (Calbiochem-Novabiochem, Germany). Recombinant baculoviruses were generated as described (Krach et al., 2000).

c. Indirect immunofluorescence microscopy. Cells were grown to confluence on cover slips, fixed with 2% formaldehyde for 20 min and washed three times with phosphate-buffered saline (PBS). After permeabilization with 0.5% Triton X-100 for 10 min and blocking for 10 min with 1% BSA solution, cells were incubated with a 1:500 dilution of either antiserum or preimmune serum for 30 min followed by incubation with a 1:1,000 dilution of indocarbocyanin-conjugated anti-rabbit immunoglobulin secondary antibody (Dianova, Germany) for 30 min. Indirect immunofluorescence for the analysis of PERV Gag or Env protein expression was performed using a laser scan microscope as described previously (Tönjes et al., 1997).

d. Immunoblotting. Sucrose gradient purified PERV particles and lysates of cell line 293 PERV-PK were analyzed by 10% SDS-polyacrylamide gel electrophoresis (SDS-PAGE) and Western blotting using polyvinylidene difluoride membranes (Millipore, Germany). Blots were incubated with a 1:1,000 dilution of antisera for 1 hour or overnight followed by a 1:10,000 dilution of protein G conjugated horseradish peroxidase (BioRad, Germany) for 1 hour. Immunoreactive proteins on membranes were detected using the ECL system and exposure for 15 to 20 sec on hyperfilm ECL (Amersham-Pharmacia, Germany).

e. Purification of PERV particles. Retroviral particles were isolated from 293 PERV-PK cell culture supernatants by sucrose cushion centrifugation. Stocks were stored for further use at −80° C.

1. A replication-competent molecular clone of porcine endogenous retrovirus (PERV), wherein said molecular clone was isolated from porcine cells and is replication-competent upon transfection into susceptible cells.

2. A replication-competent molecular clone according to claim 1, wherein said clone is a PERV-A clone.

3. A replication-competent molecular clone according to claim 2, wherein said clone is encoded by a nucleic acid sequence corresponding to SEQ ID NO:1.

4. A replication-competent molecular clone according to claim 1, wherein said clone is a PERV-B clone.

5. A replication-competent molecular clone according to claim 4, wherein said clone is encoded by a nucleic acid sequence corresponding to SEQ ID NO:2.

6. A replication-competent molecular clone of PERV-A or PERV-B, wherein said clone was isolated from a porcine bacterial artificial chromosome library.

7. A replication-competent molecular clone according to claim 6, wherein said clone is a PERV-A clone, wherein said PERV-A clone is encoded by a nucleic acid sequence corresponding to SEQ ID NO:3.

8. A replication-competent molecular clone according to claim 6, wherein said clone is a PERV-B clone, wherein said PERV-B clone is encoded by a nucleic acid sequence corresponding to SEQ ID NO:4.

9. An Env polypeptide encoded by the nucleic acid sequence of SEQ ID NOs: 1, 2, 3 or 4.

10. A Gag polypeptide encoded by the nucleic acid sequence of SEQ ID NOs: 1, 2, 3 or 4.

11. A porcine nucleic acid sequence, wherein said nucleic acid sequence is the 5′- or 3′-flanking sequence of the integration site of a replication-competent molecular clone in the porcine genome.

12. A porcine nucleic acid sequence according to claim 10, wherein said nucleic acid sequence is selected from the group consisting of:
the 5′-flanking sequence of the PERV-A clone identified by SEQ ID NO:5,
the 3′-flanking sequence of PERV-A clone identified by SEQ ID NO:6,
the 5′-flanking sequence of the PERV-B clone identified by SEQ ID NO:7,
the 3′-flanking sequence of the PERV-B clone identified by SEQ ID NO:8,
the 5′-flanking sequence of the PERV-A clone identified by SEQ ID NO:9,
and 3′-flanking sequences of the PERV-A clone identified by SEQ ID NO:10,
the 5′-flanking sequences of the PERV-B clone identified by SEQ ID NO:11,
and/or the 3′-flanking sequences of the PERV-B clone identified by SEQ ID NO:12.

13. An oligonucleotide for the detection of integrated PERVs wherein said oligonucleotide comprises 12-60 nucleotides of
the 5′-flanking sequence of the PERV-A clone identified by SEQ ID NO:5,
the 3′-flanking sequence of the PERV-A clone identified by SEQ ID NO:6,
the 5′-flanking sequence of the PERV-B clone identified by SEQ ID NO:7,
the 3′-flanking sequence of the PERV-B clone identified by SEQ ID NO:8,
the 5′-flanking sequence of the PERV-A clone identified by SEQ ID NO:9,
and 3′-flanking sequences of the PERV-A clone identified by SEQ ID NO:10,
the 5′-flanking sequences of the PERV-B clone identified by SEQ ID NO:11,
and/or 3′-flanking sequences of the PERV-B clone identified by SEQ ID NO:12
or an oligonucleotide which is complementary to one of the flanking sequences and comprises 12-60 nucleotides,
or which hybridizes to the flanking sequences and comprises 17-60 nucleotides.

14. Oligonucleotide according to claim 12, wherein the oligonucleotide comprises 20 to nucleotides.

15. A method for detecting the presence of infectious PERV particles in a sample wherein the method comprises the detection of infectious PERVs using the oligonucleotides according to claims 12 to 13.

16. A method for detecting the presence of infectious PERV particles in a sample, comprising the detection of the nucleic acid sequences of a replication-competent molecular clone of any one of claims 1 to 8.

17. A method for detecting the presence of infectious PERV particles in a sample, comprising detecting the polypeptides according to claim 9.

18. A vaccine for immunizing a host against a replication-competent PERV, comprising an effective amount of polypeptides according to claim 9.

19. A method for isolating a replication-competent molecular clone of PERV, comprising the steps of
a) establishing a DNA library from the porcine cell line PK15, wherein said cell line releases infectious PERV particles,
b) screening said DNA library with a PERV-specific pro/pol probe,
c) isolating clones containing proviral sequences which react with the PERV-specific pro/pol probe from said DNA library,
d) analyzing said proviral sequences from said DNA library with PCR employing PCR primers specific for PERV-A and PERV-B env genes, and
e) determining the presence of a proviral ORF in the isolated proviral sequences by protein truncation test (PTT).

20. A method according to claim 18, wherein after step (e), the replication-competence of the isolated clone is determined by
f) transfecting susceptible cells with the isolated clone, and
g) detecting expression and productive infection of susceptible cells by indirect immunofluorescence analysis using a PERV-specific Gag p10 antiserum and determining reverse transcriptase activity in the supernatants of the infected susceptible cells.

21. A method according to claims 19 and 20, wherein in step(a), a porcine bacterial artificial chromosome library is established from primary fibroblasts derived from large white pigs
